(19) United States
(12) Patent Application Publication
(10) Pub. No.: US 2008/0153795 A1
(43) Pub. Date: Jun. 26, 2008

(54) Medicaments and methods for wound healing

(75) Inventors: Nick Occleston, Manchester (GB); Kerry Nield, Manchester (GB); Sharon O'Kane, Manchester (GB); Mark Ferguson, Manchester (GB)

Correspondence Address: NIXON & VANDERHYE, PC, 901 NORTH GLEBE ROAD, 11TH FLOOR, ARLINGTON, VA, 22203, US

(73) Assignee: RENOVO LIMITED, Manchester (GB)

(21) Appl. No.: 11/723,193

(22) Filed: Mar. 16, 2007

(30) Foreign Application Priority Data
Dec. 23, 2006 (GB) GB 0625963.4

(51) Int. Cl. A61K 31/56; A61P 17/02

(52) U.S. Cl. 514/182

(57) ABSTRACT

The present invention relates to the manufacture of medicaments for accelerating the healing of wounds. The invention also relates to methods of treatment for accelerating the healing of wounds. In particular the invention relates to medicaments and methods for the acceleration of healing of skin wounds. The invention relates to wound dressings that are able to provide active agents to a wound, and thereby accelerate healing of the wound, and to formulations such as creams or sprays that may be used to accelerate healing.

The present invention relates to the manufacture of medicaments for accelerating the healing of wounds. The invention also relates to methods of treatment for accelerating the healing of wounds. In particular the invention relates to medicaments and methods for the acceleration of healing of skin wounds. The invention relates to wound dressings that are able to provide active agents to a wound, and thereby accelerate healing of the wound, and to formulations such as creams or sprays that may be used to accelerate healing.

The skin is the most frequently injured of the body's organs. By virtue of its location, the skin is in constant contact with the external environment, and as a result is the organ most frequently exposed to environmental, and other, damage.

Wounds, such as skin wounds, may arise as a result of many different forms of damage. Such damage may impair or entirely destroy the function of the injured organ or tissue, and the outcome of such damage depends on the nature and role of the tissue or organ affected.

The wound healing response is most commonly described with reference to the healing of skin wounds. The response involves a sequence of overlapping reparative processes. The most important of these processes in terms of influencing the rate of wound healing is that of re-epithelialisation.

Skin wounds generally re-epithelialise “from the outside in”. Keratinocytes from the unwounded skin surrounding the damaged area proliferate and migrate to cover the tissue at the wound site. This migration of keratinocytes means that the area around the edge of the wound is the first to heal, and the progress of the keratinocytes over the damaged area provides a useful index by which the progress of wound healing may be measured. In cases where epidermal appendages remain intact within the wounded area, for example, in the case of partial thickness wounds, keratinocytes involved in the re-epithelialisation response may also be derived from these structures.

Wounding of organs such as the skin gives rise to a number of undesirable effects. One noticeable effect is that the presence of a wound impairs the barrier function of the skin. This increases the rate at which fluids are lost from the wounded area, which can be a particular concern in the case of wounds covering large areas. It is common for burns victims to suffer severe, potentially life-threatening, dehydration as a result of fluid loss through the damaged skin. The loss of the skin's barrier function also increases the risk of ingress and infection by pathogens such as bacteria and fungi.

Wounds are painful, even aside from the events associated with their formation, and delays in the healing of wounds may be associated with extended incidences of pain to the sufferer. Wounds can also decrease the mechanical function of the injured area.

In the light of the above, it will be seen that the acceleration of healing of wounds is advantageous for many different reasons. However, despite the fact that the advantages of accelerating wound healing are well recognised, there remains a paucity of medicaments and methods by which this aim may be achieved. Accordingly there is a requirement for new, alternative, and more effective, medicaments and methods by which acceleration of wound healing may be attained.

There is significant variation in the range of therapies currently used to accelerate the healing of wounds. At their most basic level these may merely be concerned with preventing or stopping blood loss as a result of the wound, and preventing infection of the wound in an attempt to promote healing.

More advanced therapies known from the prior art include the use of wound management techniques, accelerants for wound closure and skin substitutes for graft augmentation. Debridement is often used for the treatment of chronic wounds to remove non-viable tissue, this usually takes the form of mechanical/surgical or enzymatic removal of the effected tissue. Surgical excision rapidly clears the area of the affected tissue but can be extremely painful, it is also non-specific and can therefore lead to complications induced by the increased injury to the area. Addition of enzymatic agents such as collagenase, papain-urea and bromelain to clear the wound area have yielded mixed efficacy in current studies.

Currently the control of the wound closure is reliant upon the initial dressing and wound management via the addition of antimicrobial agents to the wound site. A number of dressings are used to clear the site of extraneous fluid/tissue either by absorption or using vacuum-assisted closure. A common disadvantage of dressings of this nature is the need to retain the dressing in position, which may not be possible depending on wound site.

The addition of silver as an antimicrobial agent to dressings has been extensively used, with a number of products on the market, but the efficacy of these products is mixed. Skin substitutes offer a method for temporary wound coverage but ultimately auto-grafting is required due to the survival of the cultured sheets of cells.

A number of targets exist for the acceleration of wound healing, these include growth factors, oxidised regenerated cellulose/collagen matrices, adenosine A_{2A} agonists and recombinant lactoferrin. These act in a variety of ways; the collagen matrix acts to inhibit the negative impact of proteases and oxygen free radicals whilst promoting the activity of growth factors within the wound area; the lactoferrin acts to promote IL-18 up-regulation within the wound. Gene therapy is now being utilised for the treatment of impaired wound healing, with the modulation of growth factors being the primary target. This is an extremely underdeveloped field and requires a much greater level of research to investigate its efficacy.

The management of wounds, such as split thickness skin graft donor sites (reviewed in Rakel et al. 1998) or sites subject to skin peels, may merely involve leaving the graft donor site exposed and untreated, or may alternatively make use of treatments such as the application of dressings (typically gauze dressings, which may be used alone or impregnated with a variety of anti-infective agents, alginates, hydrocolloids, synthetic composite membranes, transparent films or honey), application of artificial skin (which may be generated from the individuals own epidermis), application of allografts (typically bovine or porcine allografts) or application of ointments (typically ointments containing silver based compounds as anti-infective agents).

The absence of a single universally accepted method for accelerating the healing of wounds is indicative of the need for novel medicaments and methods by which such acceleration may be effected. It is well recognised that there are failings and disadvantages associated with many of the current therapies available. Even in the case of relatively successful therapies, there is scope for improvement in terms of increased efficacy, or other parameters.

There are a number of adverse effects associated with current regimes used in the management of wounds. These include protracted healing times, which may ultimately lead to the development of chronic wounds. Other undesirable effects relate to the qualities of the replacement tissues or organs that are generated via the healing process. These may frequently be rougher and/or thinner than those originally present. Slow rates of healing may be associated with increased rates of infection. These may be increased in the case of treatments using allograft materials, which may harbour agents of infection, such as bacteria, fungi, prions or viruses. Delays in wound healing may also be associated with increased edema, erythema and pain.

One effective therapy for the promotion of re-epithelialisation of skin wounds comprises the administration to the wound of a compound that promotes oestrogenic activity.

It is an object of certain aspects of the invention to provide new medicaments and methods that may be used to accelerate the healing of wounds. It is an object of certain aspects of the invention to provide alternative medicaments and methods that may be used to accelerate the healing of wounds. It is an object of certain aspects of the invention to provide medicaments and methods that may be used to accelerate the healing of wounds with greater efficiency than is achieved by the prior art. It is an object of certain aspects of the invention to provide medicaments and methods that may be used to accelerate the healing of wounds to a greater extent than may be achieved by the prior art. It is an object of certain aspects of the invention to provide more cost effective medicaments or methods of treatment for the acceleration of wound healing.

In a first aspect, the invention provides the use of a compound that promotes oestrogenic activity in the manufacture of a medicament for administration in an amount to provide per cm² of the administered area a level of oestrogenic activity equivalent to that provided by up to 300 ng of 17β-oestradiol, for accelerating the healing of wounds. Preferably the medicament may be formulated to provide an amount of oestrogenic activity equivalent to that provided by between 1 ng and 300 ng of 17β-oestradiol per cm² of the administered area. More preferably the medicament may be formulated to provide an amount of oestrogenic activity equivalent to that provided by between 20 ng and 300 ng of 17β-oestradiol per cm² of the administered area. Even more preferably the medicament may be formulated to provide an amount of oestrogenic activity equivalent to that provided by between 100 ng and 200 ng of 17β-oestradiol per cm² of the administered area.

A medicament of the invention may be formulated to provide an amount of oestrogenic activity equivalent to that provided by 100 ng of 17β-oestradiol per cm² of the administered area.

Alternatively, a medicament of the invention may be formulated to provide an amount of oestrogenic activity equivalent to that provided by 200 ng of 17β-oestradiol per cm² of the administered area.

In a second aspect the invention provides a method of accelerating the healing of a wound, the method comprising administering to a site in need of such accelerated healing a therapeutically effective amount of a compound that promotes oestrogenic activity, the therapeutically effective amount being an amount that provides oestrogenic activity per cm² administered equivalent to that produced by up to 300 ng of 17β-oestradiol. A site in need of accelerated healing in accordance with this aspect of the invention may be a wound, or a site where a wound is to be formed. Such a site (whether a wound, or a site where a wound is to be formed) may preferably be in the skin.

17β-oestradiol represents a preferred compound that promotes oestrogenic activity suitable for use in the medicaments and methods of the invention.

The skilled person will appreciate that the methods of treatment of the invention may suitably be practiced using the medicaments of the invention.

The invention is based on the surprising finding that an amount of oestrogenic activity equivalent to that produced by up to 300 ng of 17β-oestradiol per cm² of a wound, or an area where a wound is to be formed, constitutes a therapeutically effective amount of oestrogenic activity sufficient to accelerate wound healing. This finding is highly surprising in light of the state of the art, since, although it was known prior to the present disclosure that compounds which promote oestrogenic activity may be used to accelerate the healing of wounds, it was previously believed that a far larger amount of oestrogenic activity was required to achieve this therapeutic effect.

The inventors believe that the acceleration of wound healing achieved using the medicaments or methods of the invention is mediated by an increase in the rate of re-epithelialisation of wounds exposed to a therapeutically effective amount of a compound that promotes oestrogenic activity. The inventors believe that, although this re-epithelialisation occurs primarily as a result of migration of epithelial cells from surrounding uninjured areas, the therapeutically effective amount of the compound that promotes oestrogenic activity (whether in the context of medicaments or methods of the invention) should preferably be provided to the connective tissue underlying the wound (or site where a wound is to be formed).

The inventors' studies indicate that the increase in re-epithelialisation achieved using the medicaments or methods of the invention occurs in the absence of wound contraction.

Wound contraction, particularly excessive wound contraction arising after connective tissue injuries (including skin wounds, ligament, and tendon injuries) is a major concern to both patients and physicians, since it can result in limb or joint dislocation and/or loss of function. Many wound types can be affected by detrimental wound contraction.

Detrimental wound contraction may be particularly problematic in the case of large wounds, such as those associated with grafting procedures (particularly at graft donor sites) or large burns. Accordingly these wounds may gain particular benefit from treatment with the medicaments or methods of the invention. Other types of wound are prone to detrimental wound contraction by virtue of the tissues injured. Examples of these include strictures of the duodenum following chronic peptic ulcer disease, urethral strictures and strictures of the esophagus after lye burns, and contraction following skin peels (particularly at cosmetically important sites, such as the face). It will be recognized that these wounds may also gain particular advantage through treatment using the medicaments or methods of the invention.

The inventors have shown for the first time in the present disclosure that effective acceleration of the healing of wounds may be achieved using much reduced levels of oestrogenic activity. Indeed, the inventors have most surprisingly shown that these reduced levels of oestrogenic activity are even more effective in accelerating the healing of wounds than are the medicaments and methods disclosed in the prior art.

The finding that oestrogenic activity equivalent to that provided by administration of up to 300 ng of 17β-oestradiol per centimetre of wound, is effective to accelerate the healing of wounds represents a very particular sub-range within the broader range of oestrogenic activities that the skilled person might be led to suppose would be therapeutically effective. This very narrow sub-range has not previously been identified as effective, and certainly not as providing a preferred range that provides improved therapeutic activity compared to the amounts of therapeutic activity that lie above or below this range. Thus the present disclosure is not only the first to consider wound healing medicaments providing oestrogenic activity within this specific range, but is also the first to consider the benefits provided by medicaments providing oestrogenic activity within this sub-range.

It will be appreciated that the ability to accelerate the healing of wounds using reduced amounts of compounds that promote oestrogenic activity (requiring only the promotion of oestrogenic activity equivalent to that produced by administration of up to 300 ng of 17β-oestradiol per cm² treated) in accordance with the present invention confers notable advantages in terms of reducing the costs involved in the manufacture of medicaments to be used in such treatments. It is generally the active compound (or compounds) in a medicament that contributes most to the cost of manufacturing the medicament, and hence the finding, provided by the present disclosure, that effective acceleration of the healing of wounds may be achieved using relatively lower amounts of such active compounds than previously believed, allows more economic methods of therapy to be achieved.

There is an increasing recognition of the fact that the use of compounds that promote oestrogenic activity may have damaging effects on the environment. “Oestrogenic pollution” caused by excretion of compounds that promote activity into the surrounding environment is generally believed to be responsible for a range of undesirable side-effects, including the feminization of populations of aquatic fauna. The reduced quantities of compounds that promote oestrogenic activity that are utilised in the medicaments and methods of the present invention, as compared to the amounts used in prior art compositions, may thus reduce incidences of oestrogenic pollution associated with the manufacture or use of the present medicaments.

It is preferred that the wounds, healing of which is to be accelerated by medicaments or methods of the invention, may be skin wounds. However, the inventors believe that medicaments or methods of the invention may be successfully used to promote accelerated healing in a range of tissues or organs, including the eyes (and particularly the cornea).

Skin wounds, the healing of which may be accelerated using the medicaments and methods of the invention, may be acute wounds or chronic wounds.

Particular acute wounds that may benefit from accelerated healing provided by the medicaments or methods of the invention include surgical wounds (and particularly those associated with cosmetic procedures and/or grafting procedures), wounds resulting from skin peels (or other procedures leading to the production of partial-thickness wounds), and pre-tibial lacerations.

Preferred chronic wounds that may be treated with the medicaments or methods of the invention in order to accelerate their healing include ulcers such as diabetic ulcers, decubitus ulcers, and venous ulcers.

In a preferred embodiment the medicaments of the invention may comprise solid medicaments from which a therapeutically effective amount of an active compound may be released to a wound the healing of which is to be accelerated. In particular it is preferred that a medicament of the invention may comprise a wound dressing capable of providing a therapeutically effective amount of an active compound to a wound to which the dressing has been applied. The use of solid medicaments of the invention, and particularly wound dressings of the invention, constitute preferred means by which the methods of treatment of the invention may be practiced.

In an alternative preferred embodiment, the medicaments of the invention may comprise a liquid medicament, such as a spray, cream or the like, incorporating an active compound. Such liquid medicaments may be administered to the site of a wound the healing of which is to be accelerated.

The acceleration of healing of wounds within the context of the present invention may be understood to encompass any increase in the rate of healing of a treated wound as compared to the rate of healing occurring in a control-treated or untreated wound. The rate of healing of wounds attained in accordance with the invention may readily be compared with that taking place in control-treated or untreated wounds using any suitable model of wound healing known in the art. Suitable models in which the rate of wound healing may be assessed are set out elsewhere in the specification.

Accelerated healing of a wound achieved using the medicaments or methods of the invention may preferably lead to a treated wound healing at a rate at least 5% faster than an untreated wound, preferably at a rate at least 10% faster, more preferably at least 15%, 20% or 25% faster; yet more preferably at least 50% faster, still more preferably at least 75% faster, and most preferably 100% (or more) faster. Suitable methods by which acceleration of the healing of wounds may be quantified to assess improvements in the rate of healing are described elsewhere in the specification.

Various terms that are used in the present disclosure to describe the invention will now be explained further. The definitions provided below may be expanded on elsewhere in the specification as appropriate, and as the context requires.

### “Medicaments of the Invention”

For the purposes of the present disclosure, medicaments of the invention should be taken as encompassing any medicament manufactured in accordance with any aspect or embodiment of the invention. A medicament of the invention may be suitable for putting into practice any method of treatment in accordance with the present invention. Suitable compositions, formulations and routes of delivery that may be used for medicaments of the invention are considered.

### “Active Compound”

Except for where the context requires otherwise, for the purposes of the present disclosure, an “active compound” should be taken to be any compound that promotes oestrogenic activity, and hence accelerated healing of wounds, in accordance with the present disclosure. Examples of suitable active compounds are provided elsewhere in the present disclosure, and favoured active compounds include 17β-oestradiol.

### “Therapeutically Effective Amount”

The term “therapeutically effective amount” as used in the context of the present disclosure when referring to a medicament of the invention, method of treatment of the invention, or an amount of an active compound (in accordance with the definition offered elsewhere) refers to an amount of the medicament, or of the method, or of an active compound, sufficient to provide oestrogenic activity equivalent to that produced by administration of up to 300 ng of 17β-oestradiol per cm² treated. A therapeutically effective amount of medicament, method, or active compound will be sufficient to accelerate the healing of a wound to which it is administered.

### “Promotion of Oestrogenic Activity”

In the context of the present disclosure “promotion of oestrogenic activity” may be considered to encompass any promotion or increase in oestrogenic activity that is achieved on administration of an active compound. The oestrogenic activity to be promoted may preferably be the acceleration of healing of a wound, however other activities may also be investigated in order to assess the production of a therapeutically effective amount of oestrogenic activity. It will immediately be appreciated that, once oestrogenic activity has been assessed, it is then a simple matter to determine whether or not such activity is, or has been, promoted on administration of a compound that may putatively have oestrogenic activity.

Oestrogenic activity may be assessed with reference to any one of a number of assays well known to the skilled person. Suitable assays include both in vivo and in vitro assays. For example, oestrogenic activity may be assessed in vivo by means of a rodent uterotrophic assay. Briefly, oestrogenic activity is demonstrated in such an assay by the ability of a compound to increase the weight of the uteruses of experimental rodents (such as rats). Compounds to be investigated for the ability to promote oestrogenic activity may be administered by dermal or oral routes.

Suitable in vitro assays for the assessment of oestrogenic activity may include the MCF-7 human breast cancer cell assay. In this assay oestrogenic activity of a compound is demonstrated by the ability of the compound to induce increased proliferation of the breast cancer cells.

In the case of either in vivo or in vitro assays, compounds having known oestrogenic activity, such as 17β-oestradiol, may be used as positive controls, and to produce dose response curves by which oestrogenic activity may be quantified. Such quantification may be particularly useful in assessing whether or not a compound of interest has oestrogenic activity making it suitable for use in accordance with the present invention.

### “Topical Medicament”

A “topical medicament”, for the purposes of the present disclosure, is to be construed as a medicament that is applied at a site where it is intended to have its effect. Topical medicaments suitable for use in accordance with the present invention include, but are not limited to, ointments; creams; lotions; gels; sprays; wound dressings capable of releasing active agents to the body; and injectable solutions administered by local injections (e.g. intradermal injections). Preferred routes of administration are those that allow the provision of a therapeutically effective amount of an active compound to a connective tissue (such as the dermis, in the case of skin wounds) surrounding or underlying a wound, the healing of which is to be accelerated. Topical medicaments represent preferred forms of the medicaments of the invention, and it is preferred that topical medicaments may be used to practice the methods of the invention.

Topical medicaments (i.e. those having their effect at the site, and preferably in the tissue, to which they are administered) will generally be preferred over medicaments or routes of administration associated with systemic administration of agents. For example, injectable medicaments of the invention may be for use in localised routes of administration, such as intradermal injection, rather than systemic routes of administration (such as intravenous, intraperitoneal, or subcutaneous injection).

### “Wounds”

For the purpose of the present disclosure wounds will primarily be described with reference to skin wounds, which comprise preferred wounds the healing of which may be accelerated in accordance with the present invention. However, the skilled person will appreciate that the acceleration of healing of wounds in accordance with the invention should preferably not be limited to skin wounds. The inventors believe that healing of wounds may be accelerated in wounds of all tissues, though these will preferably be of tissues other than those of the urinogenitary organs (here considered to comprise in particular the organs of the reproductive tract and the bladder).

Wounds, the healing of which may be accelerated using medicaments or methods of the invention, should involve an either total or partial breakdown of an epithelial layer.

Skin wounds, the healing of which may be accelerated using the medicaments and methods of the invention, include both chronic wounds and acute wounds. Examples of suitable chronic or acute wounds the healing of which may be accelerated in accordance with the invention are set out elsewhere in the specification.

The medicaments and methods of the invention may be used to accelerate the healing of full thickness or partial thickness wounds (respectively wounds in which the epithelial layer is either totally or partly compromised). Preferred examples of partial thickness wounds the healing of which may be accelerated using the medicaments or methods of the invention include “skin peels” such as “chemical peels” (such as alphahydroxy acid peels, trichloroacetic acid peels or phenol peels) or “laser peels”; wounds associated with dermabrasion; wounds associated with dermaplaning; wounds associated with photorefractive keratectomy (PRK); and wounds associated with laser tattoo removal.

Examples of specific wounds, other than those of the skin, which may benefit from accelerated healing of wounds in accordance with the present invention include, but are not limited to, those selected from the group consisting of: wounds of the eye (including wounds of the cornea, and acceleration of healing of wounds resulting from eye surgery such as LASIK or PRK surgery); wound of blood vessels; wounds of the peripheral or central nervous system (where increasing the rate of healing of wounds may enhance the capability for neuronal reconnection); wounds of tendons, ligaments or muscle; and wounds of the oral cavity, including the lips and palate; wounds of the internal organs such as the liver, heart, brain and digestive tissues; and wounds in body cavities such as the abdominal cavity, pelvic cavity and thoracic cavity.

Preferred wounds, the healing of which may be accelerated using the medicaments and methods of the invention, include chronic wounds, pre-tibial lacerations, wounds associated with skin peels (or other procedures producing partial thickness wounds) and wounds of the immunocompromised.

It is particularly preferred that the medicaments and methods of the invention be used to accelerate the healing of skin wounds.

### “Accelerating the Healing of Wounds”

“Acceleration of the healing of wounds”, or “acceleration of wound healing” in the context of the present disclosure should be taken to encompass any increase in the rate at which a wound is covered, and so healed.

The inventors have found that the medicaments or methods of the invention are able to increase the rate of re-epithelialisation of treated wounds, and an acceleration of wound healing may be most readily demonstrated by an increased rate of re-epithelialisation of a wound. Such an increase in the rate at which the epithelial covering (for example the epidermis) is repaired or regenerated will indicate that the healing of the wound in question has been accelerated.

An increase in the rate of re-epithelialisation of a wound should preferably be distinguished from closure of a wound brought about by wound contraction. In this case contraction of elements (generally thought to be myofibroblasts) located within and around the periphery of the wound leads to a reduction in the surface area of the wound, but this may not be associated with covering of the wound by epithelium.

Accelerated healing of wounds in the context of the present disclosure may also be distinguished from “filling” of wounds that may occur through the generation of granulation tissue.

Accelerated healing of a wound in accordance with the present disclosure may preferably lead to a treated wound healing at a rate at least 5% faster than an untreated wound, preferably at a rate at least 10% faster, more preferably at least 15%, 20% or 25% faster; yet more preferably at least 50% faster, still more preferably at least 75% faster, and most preferably 100% (or more) faster.

Accelerated healing of a wound in accordance with the present disclosure may preferably lead to a treated wound having a “healing age” that is at least a day faster than an untreated wound, preferably at a rate at least five days faster, more preferably at least ten days faster; yet more preferably at least eleven, twelve, thirteen, fourteen of fifteen days faster, still more preferably fifteen or more days faster, and most preferably 20 (or more) days faster.

### “Treated Wounds”, “Control-Treated Wounds” and “Untreated Wounds”

A “treated wound” in the context of the present disclosure is any wound that has been provided with a therapeutically effective amount of a medicament of the invention, or a therapeutically effective amount of an active compound administered in accordance with a method of treatment of the invention.

“Control-treated wounds” and “untreated wounds” in the present context are respectively wounds treated with a relevant control, and wounds that have not been treated before, or during, healing. Control wounds will not be treated with a medicament of the invention, and preferably will not be treated with a therapeutically effective amount of an active compound. That said, wounds treated with medicaments known from the prior art may constitute suitable control wounds for comparative purposes (for example to illustrate increased efficiency or effectiveness of medicaments of the invention as compared to those already known).

It will be appreciated that untreated wounds will be expected to have a rate of healing that is not accelerated, while control wounds may be subject to either accelerated or retarded healing depending on the activity of the control administered.

### “Centimetre of Administered Area” and “Centimetre of Wound”

A “cm² of administered area” in the context of the present disclosure is a square centimetre of a human or animal tissue to which a medicament of the invention has been administered, or to which a therapeutically effective amount of a compound that promotes oestrogenic activity has been administered in accordance with the methods of treatment of the present invention. A cm² of administered area may comprise a wound, or a site where a wound is to be formed.

A “wound centimetre”, “centimetre of wound” or “centimetre of wounding” in the context of the present disclosure constitutes a unit by which the size of a wound to be treated may be measured.

A wound centimetre may be taken to comprise any square centimetre of a body surface that is wounded in whole or in part. For example, a wound of two centimetres length and one centimetre width (i.e. with a total surface area of two cm²) will also be considered to constitute “two wound centimetres”, while a wound having a length of two centimetres and a width of two centimetres (i.e. a total surface area of four cm²) will constitute four wound centimetres. By the same token, a linear wound of two centimetres length, but of negligible width (i.e. with negligible surface area), will, for the purposes of the present invention, be considered to constitute “two wound centimetres”, if it passes through two square centimetres of the body surface.

The size of a wound in wound centimetres should generally be assessed when the wound is in its relaxed state (i.e. when the body site bearing the wounded area is in the position adopted when the body is at rest). In the case of skin wounds, the size of the wound should be assessed when the skin is not subject to external tension.

An inch of administered area, or inch of wound, may be similarly defined, save that the relevant units of length or area are measured in inches rather than centimetres. It will be appreciated that the quantity of a compound that promotes oestrogenic activity constituting a therapeutically effective amount for the treatment of an inch of wound may be derived from those quantities provided in respect of centimetres of wound by use of a suitable conversion factor (one inch corresponding to approximately 2.54 centimetres).

A centimetre or inch of wounding may thus provide a unit by which the size of a wound to be treated may be measured, and the required amount of a medicament of the invention (or of an active compound administered in accordance with a method of treatment of the invention) may be determined.

As noted above, the skin suffers from more direct, frequent, and damaging encounters with the external environment than any other organ in the body. As a result the skin suffers from more wounds than other organs, and it is therefore highly desirable to be able to accelerate the healing of skin wounds in order to return this organ as rapidly as possible to its maximum functional effectiveness. Acceleration of the healing of skin wounds is a preferred embodiment of the medicaments or methods of the present invention. Skin wounds susceptible to accelerated healing in accordance with the medicaments or methods of the invention include both “open” wounds, in which the integrity of the skin has been entirely compromised, exposing the underlying tissues, and also “closed” wounds in which the skin, though damaged, is not entirely compromised. Partial thickness wounds, as described elsewhere in the specification (particularly in the context of skin peels and skin grafts), provide an example of closed wounds that may benefit from accelerated healing as provided by the invention.

Accelerating healing of skin wounds in accordance with the invention is able to hasten the formation of a functioning protective barrier over previously damaged or denuded areas. The accelerated healing helps prevent ingress into, and colonization of, the underlying tissue by pathogens such as bacteria, fungi and viruses. Thus accelerating the healing of wounds using the medicaments or methods of the invention may provide benefits in contexts in which it is desirable to prevent or reduce infection of wounds.

Healed skin (and in particular the re-established intact epidermis) also acts as a barrier to fluid movement, and is therefore able to prevent desiccation of underlying tissue. Thus accelerated wound healing that may be achieved with the medicaments or methods of the invention may help to prevent or reduce tissue desiccation arising as a result of fluid loss across damaged areas of the skin. As set out elsewhere in the specification, the damage caused as a result of fluid loss through wounded skin is particularly damaging in the case of wounds having large surface areas, such as burns or skin peels. The acceleration of the healing of burns wounds or wounds associated with skin peels thus represents a preferred application of the methods or medicaments of the invention.

It will be appreciated that accelerated healing of wounds that may be achieved by the medicaments and methods of the invention may be of particular benefit in cases in which the wound healing response is impaired, inhibited, retarded or otherwise defective as compared to the normal rate of healing. The methods and medicaments of the invention may also be used to accelerate the healing of wounds in patients that are not subject to an impaired healing response. Illustrative examples of both contexts are set out below.

There are many contexts in which the body's healing response is defective and may benefit from acceleration using the medicaments or methods of the invention. These include conditions such as pemphigus, Hailey-Hailey disease (familial benign pemphigus), toxic epidermal necrolysis (TEN)/Lyell's syndrome, epidermolysis bullosa, cutaneous leishmaniasis and actinic keratosis. Retarded healing of wounds of the eye may be associated with conditions such as partial limbal stem cell deficiency or corneal erosions.

Healing of wounds may also be retarded as a result of the actions of pathogens (such as bacteria, fungi or viruses), chemical insults (such as chemical burns caused by caustic agents, or through the effect of cytotoxic drugs such as those employed in chemotherapy), or as a result of radiation damage (either through particulate radiation or electromagnetic radiation such as gamma radiation, ultraviolet radiation, or the like) such as that occurring in sunburn. Accordingly wounds subject to any of these influences may be particularly suitable subjects for acceleration of healing using the medicaments or methods of the invention.

It is well known that dermal injuries in the aged heal more slowly than do those of younger individuals. The aged may therefore particularly benefit from accelerated healing brought about by the medicaments and methods of the invention. There are also many other conditions or disorders that are associated with a delayed or otherwise impaired wound healing response. For example patients with diabetes, patients with polypharmacy (for example as a result of old age), post-menopausal women, patients susceptible to pressure injuries (for example paraplegics), patients with venous disease, clinically obese patients, patients receiving chemotherapy, patients receiving radiotherapy, patients receiving steroid treatment or immuno-compromised patients may all suffer from impaired epithelial regeneration. In some cases the slower healing response exhibited by such patients may contribute to the development of infections at the site of wounds. The slow wound healing response may also be associated with the formation of chronic wounds, as considered below. Accordingly, it will be appreciated that such patients represent a preferred group that may benefit from accelerated wound healing using the methods or medicaments of the invention.

The inventors believe that the medicaments and methods of the invention may be of particular value to aged or senescent patients. Aged or senescent patients, for the purposes of the present disclosure, may be defined as comprising patients aged 65 years or older, more preferably aged 75 years or older. In the case of female patients, it may be preferred that the medicaments or methods of the invention be provided to post-menopausal patients, to whom they may be of marked benefit.

Without detracting from the above, it may generally be preferred that the medicaments or methods of the invention may be utilised to accelerate the healing of wounds of patients not subject to delayed wound healing. Acceleration in this way will give rise to a faster wound healing response than would normally be achieved by such patients in the absence of therapeutic acceleration (i.e. give rise to faster healing than in control wounds). Accordingly the wounds of such patients may be induced to heal more rapidly.

The skilled person will immediately appreciate, that there is a great benefit to be gained by society from the development of therapeutic agents and techniques that can hasten the healing of otherwise healthy patients. As well as the various benefits considered elsewhere in the specification, accelerating healing in this manner can help reduce time spent in convalescence, and can thus benefit productivity. Accordingly, the acceleration of healing of wounds of healthy patients is a preferred embodiment of all aspects of the present invention.

The medicaments and methods of the invention may be used to accelerate the healing of both chronic wounds and acute wounds. For the purposes of the present invention, a chronic wound may be defined as any wound that does not show any healing tendency within eight weeks of formation when subject to appropriate (conventional) therapeutic treatment. Acute wounds may be any wound other than a chronic wound.

Acceleration of the healing of chronic wounds is a preferred embodiment of the invention. Examples of chronic wounds that may benefit from accelerated healing provided by the medicaments or methods of the invention may be selected from the group comprising: leg ulcers; venous ulcers; diabetic ulcers; bed sores; decubitus ulcers; foot ulcers; and pressure ulcers. It will be appreciated that the long lasting nature of chronic wounds exacerbates many of the disadvantages associated with normal wound healing. For example, the duration of the period over which a patient suffering from a chronic wound will experience pain will generally be far longer than for a patient with an acute wound. Similarly the length of time over which desiccation as a result of liquid loss may occur will also be extended. Incidences of wound infection are also much increased in chronic, as opposed to acute, wounds.

Chronic wounds are also subject to many disadvantages that are not generally associated with acute wounds. For example, chronic wounds frequently expand beyond the limits of the original wounded area. This may arise as a result of infection (which may increase the damage around the margins of the wound, thereby leading to expansion) or through maceration of the tissue surrounding the wound (typically as a consequence of increased liquid loss through the chronic wound). The propensity for chronic wounds to expand beyond the boundary of the original injury means that such wounds are frequently of great surface area. Since accelerated wound healing utilising the medicaments and methods of the invention primarily occurs from “the outside in”, it will be appreciated that this will act to counter the progression of chronic wounds, use of the medicaments and methods of the invention may be of notable benefit in the treatment of chronic wounds.

Pretibial lacerations are acute wounds of the leg that are very frequently slow to heal, and which frequently give rise to the development of leg ulcers. Existing treatments used for pretibial lacerations include the use of surgical procedures (such as the use of skin grafts and flaps) in an attempt to heal the wound before chronic wound development. Pretibial lacerations constitute acute wounds that may particularly benefit from treatment with the medicaments and methods of the invention, in order to accelerate healing and thereby reduce incidences of chronic wound formation.

The promotion of re-epithelialisation of acute wounds (as opposed to chronic wounds) is also a preferred embodiment of all aspects of the invention. Acute wounds, the healing of which may be accelerated using the medicaments and methods of the invention, include: abrasions; avulsions; crush wounds; incisional wounds; lacerations; punctures; and missile wounds, all of which may be suffered by the skin (among other tissues or organs).

Abrasions are also commonly referred to as “scrapes”. Abrasions occur as a result of the skin being rubbed away by friction against another rough surface. Common examples of abrasions include rope burns and skinned knees. An abrasion may macroscopically appear as lines of scraped skin, possibly including tiny spots of bleeding.

Avulsions occur when an entire bodily structure, or a part of such a structure, is forcibly pulled away from its site. Examples of avulsions include the loss of a permanent tooth or an ear lobe. Avulsions may, for example, arise as a result of explosions, gunshots, and animal bites. An avulsion may characteristically exhibit heavy, rapid bleeding, as well as a noticeable absence of tissue.

Crush wounds typically occur as a result of a heavy object falling onto an individual (or part of an individual). The force thus generated may split the skin and shatter or tear underlying structures. A crush wound may have irregular margins, similar in appearance to those of a laceration; however, the wound will generally be deeper and trauma to underlying muscle and bone may be apparent

Incisional wounds are also commonly referred to as “cuts”. Incisional wounds result from incision, or slicing, of a tissue with a sharp instrument, which results in a wound with relatively even edges. Incisional wounds can vary greatly in their severity, from minimal wounds (such as a paper cut) to significant wounds such as those arising as a result of surgical incision. An incisional wound may have little or profuse bleeding depending on the depth and length of the wound, and also on the tissue involved. The even edges of incisional wounds will generally readily line up, which may facilitate closure of such wounds.

Lacerations are also frequently referred to a “tears”. These wounds arise as a result of forcible separation of a tissue or organ, which will normally produce a wound having characteristic ragged edges. Lacerations are generally produced by the action of great mechanical forces against the body, either from an internal source as in childbirth, or from an external source like a punch. The laceration arises when the force exerted on a tissue or organ becomes too great for the tissue or organ to bear. A laceration may exhibit little or profuse bleeding, in much the same manner as an incisional wound. In contrast to incisional wounds however, the tissue damage is generally greater and the wound's ragged edges do not line up so readily.

Punctures are deep, narrow wounds. Punctures may typically be produced by sharp objects such as nails, knives, and broken glass being driven into the body. The depth of a puncture wound will generally be greater than its length. As a consequence there is generally little bleeding around the outside of the wound although more bleeding may occur inside the wound. This may lead to discoloration around the puncture wound.

Missile wounds are also known as “velocity wounds”. Missile wounds are caused by an object entering the body at a high speed, typically a bullet. A missile entry wound may be accompanied by an exit wound, and bleeding may be profuse, depending on the nature of the injury.

Incisional wounds constitute preferred acute wounds, the healing of which may be accelerated by the medicaments and methods of the invention. Surgical incisional wounds may constitute a particularly preferred group of acute wounds to be treated in accordance with the invention.

It will be appreciated that tissues other than the skin, such as the cornea, may also be subject to wounds of the type described above and elsewhere in the specification. Such wounds may also benefit from the acceleration of healing that is provided by use of medicaments and methods of the invention.

Burn wounds are a further class of wounds the healing of which may be accelerated using the medicaments and methods of the invention. For the purposes of the present disclosure, burns may, except for where the context requires otherwise, be considered to include tissue damage resulting from exposure to either high or low temperature, chemical agents, or radiation.

Wounds arising as a result of burns may extend over great areas of an individual so afflicted. As a result burn wounds are particularly susceptible to complications such as infection and desiccation. It will be appreciated that burns wounds may therefore derive particular benefit from the accelerated wound healing that may be brought about by the medicaments and methods of the invention.

The use of the medicaments and methods of the invention to accelerate the healing of wounds associated with skin grafting procedures represents a preferred embodiment of the invention. It is a preferred embodiment of the medicaments and methods of the invention to accelerate “normal” rates of healing of wounds associated with grafting procedures (i.e. to accelerate the healing of grafting wounds in patients without an impaired healing response).

It is another preferred aspect of the invention to accelerate the healing of grafting wounds in patients subject to impaired or reduced healing responses. Grafting may frequently be used in an attempt to promote the healing of other wounds (particularly chronic wounds) in patients subject to impaired healing, and it may be desired to accelerate the healing of wounds associated with the graft in order to reduce the likelihood of these developing adverse complications.

The accelerated healing conferred by the medicaments and methods of the invention is of benefit at the graft recipient site, and at the graft donor site. The methods and medicaments of the invention may be used to accelerate healing of wounds associated with both full and partial thickness skin grafts. Such skin grafts (i.e. either full or partial thickness grafts) may be either meshed or unmeshed.

At the graft recipient site the accelerated healing provided by the medicaments and methods of the invention is able to improve and accelerate integration of the grafted tissue. Since the beneficial effects of the medicaments and methods of the invention are believed to come about as a result of effects of the active agents on the connective tissue underlying the wounded site it will be appreciate that these advantageous effects may be available not only in the case of grafts utilising skin, but also in the case of grafts using artificial skin, or skin substitutes (since provision of the active agent to the dermis beneath the wounded site induces keratinocytes from the surrounding unwounded skin to migrate into the wounded area and thereby encourage integration of the graft).

The acceleration of wound healing graft donor sites decreases the time taken to restore a functioning skin barrier layer, and consequently reduces the potential for donor site infection. The accelerated healing also decreases incidences of blistering and tissue breakdown that may otherwise occur at the donor site.

Since wounds treated in accordance with the present invention heal faster the period over which a patient experiences pain associated with sites where the skin has been damaged or removed is reduced. Thus by accelerating the healing of wounds at such sites using the medicaments and methods of the invention it is possible to reduce the pain associated with the taking of skin grafts.

Treatment of wounds associated with skin peels (or other procedures, such as dermabrasion, that lead to the formation of partial thickness skin wounds) using the medicaments or methods of the invention represents a preferred embodiment. It will be appreciated that such treatments, when provided after the completion of the procedure in question, will promote re-epithelialisation of the wounds in question, and thus accelerate their healing.

A further advantage of accelerated healing of wounds at skin donor sites is that this decreases the time required until re-harvesting of tissue from the donor site can take place. By “re-harvesting” is meant the subsequent removal of further graftable skin from a previously used donor site. This is particularly advantageous in situations where the skin available for harvesting is limited and/or the area of skin required to be harvested is large. Examples of such situations include occasions when it is necessary to take grafts from children and/or patients suffering from burns covering a large percentage of the body surface. In these cases there may be relatively little unwounded skin that may serve as a donor site for graft material, and it may be desirable to obtain multiple donations from the sites available.

Graft donor sites may generally be treated with the methods or medicaments of the invention before the graft tissue is taken. This allows the donor site to be “primed” in advance of wounding. Such priming may further help to accelerate wound healing by avoiding any “lag” that may otherwise occur between the graft being taken and a medicament administered. Prophylactic use in this manner may further help to avoid onset of complications that may otherwise arise at the graft donor site, such as chronic wound development. In general it may be preferred to avoid the prophylactic treatment of wounds associated with skin peels or dermabrasion, since the priming of such wounds may detract from the effect achieved by the procedure in question.

It will be appreciated that the medicaments and methods of the invention may be used prophylactically in situations other than grafting procedures, such as before surgery or when there is a risk of a wound occurring through other means. Prophylactic use in this manner may be of benefit to both healthy and healing-impaired patients. In the case where the individual to be wounded may otherwise be subject to retarded or incomplete wound healing it will generally be preferred that the medicaments or methods of the invention are administered as soon as the risk of a poor rate of wound healing has been recognised.

The inventors have found that accelerated healing of wounds in accordance with the invention may be brought about using all compounds that promote oestrogenic activity tested to date. The inventors believe that suitable compounds that promote oestrogenic activity may be selected from the group consisting of: oestrogens; oestrogen receptor agonists such as ethinylyoestradiol, dienoestrol, mestranol, oestradiol, 17β-oestradiol, oestriol, conjugated oestrogens, piperazine oestrone sulphate, stilboestrol, fosfesterol tetrasodium, polyestradiol phosphate and tibolone; inhibitors of oestrogen or oestrogen receptor agonist breakdown; phytoestrogens; modulators of luteinising hormone and chorionic gonadotrophin. As set out above, 17β-oestradiol constitutes a preferred compound that promotes oestrogenic activity to be used in the medicaments and methods of the invention.

The medicaments of the invention should be taken to encompass any composition, material or device from which a compound that promotes oestrogenic activity may be provided to a wound in a therapeutically effective quantity (as defined elsewhere in the specification).

Examples of suitable compositions that may be utilised as medicaments of the invention include: creams, emulsions, ointments, irrigation solutions, sprays, foams, powders, gels, wound dressings, microneedles, liposomes, nanomicelles, thermosetting gels, microparticles, nanoparticles, crystals, biomaterials, stents, films and products suitable for use as artificial skin.

Medicaments of the invention, such as irrigation fluids of the invention, may additionally comprise one or more agents independently selected from the group consisting of: cleansers; antibiotics; antifungal agents; antiseptic agents; and anaesthetic agents. Medicaments of this sort may be of particular value in the treatment of cavitating and/or chronic wounds.

Examples of suitable materials that may be used as medicaments of the invention include: fabrics (such as woven and non-woven materials) including bandages, sticking plasters, patches, swabs, or other wound dressings.

Examples of wound dressing materials that may be used in the production of medicaments in accordance with the present invention may include dressings in the following broad classes.

“Passive products”, comprising traditional dressings that provide cover over the wound (such as gauze and tulle dressings)

“Interactive products” comprising polymeric films and forms which are mostly transparent, permeable to water vapour and oxygen, non-permeable to bacteria (hyaluronic acid, hydrogels, foam dressings)

“Bioactive products” comprising dressings that are particularly suitable for the delivery of substances active in wound healing, such as compounds that promote oestrogenic activity. Examples of bioactive products include hydrocolloids, alginates, collagens and chitosan, and these constitute preferred examples of materials that may be used in the medicaments of the invention.

Suitable examples of wound dressings that may be used in the medicaments of the invention may be selected from the following:

### “Gauze Dressings”

Gauze dressings can stick to the wound surface and disrupt the wound bed when removed. As a result gauze dressings will generally only be used on minor wounds or as secondary dressings.

### “Tulle Dressings”

Tulle dressings do not stick to wound surfaces. They are suitable for use in flat, shallow wounds, and are useful in patients with sensitive skin. Examples of tulle dressings known from the prior art include Jelonet® and Paranet®.

### “Film Dressings” and “Semipermeable Film Dressings”

Film dressings and semipermeable film dressings comprise sheets of materials that may be used to cover wounds. Such dressings may preferably comprise sterile materials. Suitable materials from which such films may be manufactured include polyurethane and chitin. Film dressing (or semipermeable film dressings) may be coated with adhesives, such as acrylic adhesives, in order to assist their retention at sites where they are required. Dressings of this type may be transparent, and therefore allow the progress of wound healing to be checked. These dressings are generally suitable for shallow wounds with low exudate. Examples known from the prior art include OpSite®, Tegaderm®

### “Hydrocolloid Dressings”

Hydrocolloid dressings may comprise carboxymethylcellulose, gelatin, pectin, elastomers and adhesives that turn into a gel when exudate is absorbed. This creates a warm, moist environment that promotes debridement and healing. Depending on the hydrocolloid dressing chosen they may be suitable for use in wounds with light to heavy exudate, sloughing or granulating wounds. Dressings of this sort are available in many forms (adhesive or non-adhesive pad, paste, powder) but most commonly as self-adhesive pads. Examples known from the prior art include DuoDERM®, Tegasorb®

### “Hydrogel Dressings”

Hydrogel dressings are composed mainly of water in a complex network or fibres that keep the polymer gel intact. Water is released to keep the wound moist. These dressings may be used for necrotic or sloughy wound beds to rehydrate and remove dead tissue. They should not be used for moderate to heavily exudating wounds. Examples known from the prior art include Tegagel®, Intrasite®

### “Alginate Dressings”

Alginate dressings are composed of calcium alginate (a seaweed component). When in contact with wound, calcium in the dressing is exchanged with sodium from wound fluid and this turns dressing into a gel that maintains a moist wound environment. These dressings are good for exudating wounds and help in debridement of sloughing wounds. In general they should not be used on low exudating wounds as this will cause dryness and scabbing. Alginate dressing should be changed daily. Examples known from the prior art include Kaltostat®, Sorbsan®

### “Polyurethane or Silicone Foam Dressings”

These dressings are designed to absorb large amounts of exudates. They maintain a moist wound environment but are not as useful as alginates or hydrocolloids for debridement. In general they should not be used on low exudating wounds as this will cause dryness and scabbing. Examples known from the prior art include Allevyn®, Lyofoam®

### “Collagen Dressings”

Collagen dressings are generally provided in the form of pads, gels or particles. They promote the deposit of newly formed collagen in the wound bed, and absorb exudate and provide a moist environment.

Suitable dressings that may be used as, or in, solid medicaments in accordance with the invention include foam or sponge dressings (i.e. foams in which the matrices in which the pores are located are solid, as opposed to liquid foams considered elsewhere in the disclosure). Such foam or sponge dressings should be capable of providing a therapeutically effective amount of a compound that promotes oestrogenic activity to a wound to which the dressing is applied. Many materials from which suitable foam dressings may be manufactured are known to those skilled in the art, and these include polyvinyl alcohol (PVA) and other similar materials.

Compounds capable of promoting oestrogenic activity may be incorporated in, or applied to, foams or sponges for use in wound dressings by any suitable technique known in the art. For example, suitable foams or sponges may be coated with compounds that promote oestrogenic activity, or impregnated with such compounds. It will be appreciated that the important concern is merely that the oestrogenic compound be incorporated in, or applied to, the foam or sponge in such a manner that a therapeutically effective amount of the compound that promotes oestrogenic activity to a treated wound.

The skilled person will be aware that a suitable wound healing dressing to be used on a particular wound may be selected with reference to the type of the wound, size of the wound, and healing progression of the wound. Generally, the selection of an appropriate wound dressing may be determined with reference to Table 1.

TABLE 1

| Wound type | Dressing type |
|---|---|
| Clean, medium-to-high | Paraffin gauze |
| exudate (epithelialising) | Knitted varicose primary dressing |
| Clean, dry, low exudate | Absorbent perforated |
| (epithelialising) | plastic film-faced dressing |
| | Vapour-permeable adhesive film dressing |
| Clean, exudating | Hydrocolloids |
| (granulating) | Foams |
| | Alginates |
| Slough-covered | Hydrocolloids |
| | Hydrogels |
| Dry, necrotic | Hydrocolloids |
| | Hydrogels |

The medicaments or methods of the invention may make use of a number of different types of wound dressings in which compounds that promote oestrogenic activity are incorporated in a dressing material such that a therapeutically effective amount of the compound that promotes oestrogenic activity is releasable to a wound.

Suitable examples may include: nonresorbable gauze/sponge dressing for external use (typically a sterile or nonsterile device intended for medical purposes, they may be placed directly on a patient's wound to absorb exudate, and are generally made from open woven or nonwoven mesh cotton cellulose or a simple chemical derivative of cellulose); hydrophilic wound dressing (again, may be sterile or nonsterile in form, are intended to cover a wound and to absorb exudate, and are manufactured of nonresorbable materials such as cotton, cotton derivatives, alginates, dextran, and rayon that have hydrophilic properties and are capable of absorbing exudate); occlusive wound dressing (which are nonresorbable, sterile or nonsterile device intended to cover a wound and thus provide or support a moist wound environment, while allowing the exchange of gases such as oxygen and water vapor through the device, and which tend to be manufactured from synthetic polymeric materials, such as polyurethane, with or without an adhesive backing); and hydrogel wound and burn dressing (available in sterile or nonsterile forms that are used to cover a wound, and thereby to absorb wound exudate, to control bleeding or fluid loss, and to protect against abrasion, friction, desiccation, and contamination; they tend to be manufactured from nonresorbable matrices, such as those made of hydrophilic polymers or other material in combination with at least 50% water). It will be appreciated that the incorporation of compounds that promote oestrogenic activity in such dressings will generally lead to their classification as “interactive dressings” under present FDA guidelines.

In the case of a medicament of the invention comprising a solid material it may be preferred that the medicament be formulated such that a predetermined area of the medicament provides a therapeutically effective amount of a compound that promotes oestrogenic activity to a wound to which the solid material is applied.

By way of example, a solid medicament may be formulated such that each square centimetre of the medicament provides an amount of oestrogenic activity equivalent to that produced by between 20 ng and 300 ng of 17β-oestradiol. It will be appreciated that, in the case that it is desired to administer 17β-oestradiol as the active compound, such a medicament may, therefore, be formulated such that it provides between 20 ng and 300 ng of 17β-oestradiol per square centimetre of the medicament.

In the case of a liquid medicament it may be preferred that the medicament is administered in sufficient quantity to provide the specified therapeutically effective amount of oestrogenic activity.

It may be preferred that medicaments of the invention are formulated to provide discrete dosage units capable of providing a specified amount of oestrogenic activity (this specified amount of oestrogenic activity will generally be a known fraction or multiple of a therapeutically effective amount of the oestrogenic activity promoted by a chosen compound). For instance dosage units may be formulated with reference to the size of a wound to be treated. It may be envisioned that medicaments in accordance with this embodiment of the invention will be formulated so that a specified quantity (for instance a specified volume or a specified weight) of the medicament may be administered to a specified size of wound (for instance a specified area of wound or a specified length of wound) in order that a therapeutically effective amount of an active compound may be administered to the wound. For instance, a suitable medicament may be formulated so that a specified quantity of the medicament (for example 0.1 nL to 100 mL of the medicament, or 0.1 ng to 100 grams of the medicament) provides an amount of oestrogenic activity equivalent to that produced by up to 300 ng of 17β-oestradiol. Suitable quantities of the medicament may preferably provide an amount of oestrogenic activity equivalent to that produced by between 1 ng and 300 ng of 17β-oestradiol, more preferably an amount equivalent to that produced by between 20 ng and 300 ng of 17β-oestradiol, and even more preferably an amount equivalent to that produced by between 100 ng and 200 ng of 17β-oestradiol. In the event that the medicaments of the invention are to be used to treat an existing wound, it may be preferred that the specified quantity of the medicament be provided per centimetre of wounding.

Suitable specified quantities of medicaments of the invention may be selected with respect to the nature of the medicament in question. By way of example, it may be preferred that the quantity of an injectable solution medicament of the invention required to administer a therapeutically effective amount of an active compound may relatively small (for example in the region of 0.05 to 0.5 mL). In contrast, the quantity of a gel, ointment or spray medicament of the invention required to administer a therapeutically effective amount of an active compound may be relatively larger (for example in the region of 0.25 to 2.5 mL). The quantity of an irrigation fluid medicament of the invention required to administer a therapeutically effective amount of an active compound may be larger still (for example in the region of 0.5 to 5 mL, or more).

It will be appreciated that the concentration of active compounds within the different forms of the medicaments of the invention may be selected, for example in accordance with the volumes outlined above, in order to provide suitable medicaments of the invention capable of providing therapeutically effective amounts of an active compound. Thus, in the case that it is wished to deliver a therapeutically effective amount of up to 300 ng of 17β-oestradiol, this amount of the active compound may be incorporated in a volume of between 0.05 and 0.5 mL of an injectable solution, in a volume of between 0.25 and 2.5 mL of a gel, ointment or spray, and in a volume of between 0.5 and 5 mL, or greater, of an irrigation fluid. Suitable concentrations of other active compounds that may be used in the medicaments of the invention will be readily apparent to those skilled in the art.

Medicaments of the invention may be provided in the form of discrete dosage units capable of providing a therapeutically effective amount of an active compound (which is to say capable of providing an amount of a medicament sufficient to provide a therapeutically effective amount of oestrogenic activity). A suitable dosage unit in accordance with this embodiment of the invention may comprise a sufficient amount of a medicament of the invention to accelerate the healing of a given length or area of a wound.

A suitable dosage unit may comprise sufficient of a medicament of the invention to accelerate the healing of one centimetre of a wound. Alternatively a suitable dosage unit may comprise sufficient of a medicament of the invention to accelerate the healing of one inch of a wound. It will be appreciated that medicaments of the invention may be formulated to provide single dosage units or to provide multiple dosage units, as required. Thus a medicament of the invention may be packaged to provide one or more dosage units. Each dosage unit may provide a known fraction or multiple of a therapeutically effective amount of a compound that promotes oestrogenic activity.

Suitable forms in which such discrete dosage units may be provided can be selected with reference to the nature of the medicament to be administered. Merely by way of example, medicaments of the invention comprising injectable solutions may be provided in the form of vials or pre-filled syringes comprising one or more dosage units. Other liquid medicaments in accordance with the invention, such as gels, creams, ointments, irrigation fluids or the like, may be provided in the form of tubes, sachets, cartons or blister packs comprising one or more dosage units.

Solid medicaments of the invention may readily be formulated such that a given area of the solid medicament is capable of providing sufficient of an active compound (i.e. sufficient therapeutically effective oestrogenic activity) to heal a matching-sized area of wound. In such an embodiment a solid medicament of the invention may be cut to the required size and/or shape to cover a wound, and the medicament will release a therapeutically effective amount of an active compound into the connective tissue underlying the wound and thereby accelerate the healing of the wound.

Furthermore, since it will be appreciated that the therapeutically effective amounts of oestrogenic activity provided by the medicaments of the invention are so low that they will generally not be detrimental to unwounded tissue, the skilled person will appreciate that a solid medicament, formulated such that a given area of the medicament can provide sufficient oestrogenic activity to accelerate healing of a corresponding area of wound, may be placed over an area that includes both wounded and unwounded tissue without the need to shape the medicament such that it conforms to the wounded area alone. Contact between the medicament and the wounded area covered will ensure that a therapeutically effective amount of an active compound will be provided to the wound, thereby accelerating its healing, while the amount of oestrogenic activity provided to the unwounded area will generally be insufficient to induce adverse effects.

Medicaments of the invention, such as solid medicaments, may be applied to a tissue prior to wounding, such that a therapeutically effective amount of the compound that promotes oestrogenic activity is provided to the connective tissue underlying the tissue to be wounded. Use of the medicaments of the invention in this manner will allow the site where a wound is to be formed to be “primed”, prior to wounding, to give rise to accelerated healing.

The period of time over which this therapeutically effective amount of a compound that promotes oestrogenic activity is administered may be selected with reference to the nature of the medicament. For example medicaments intended to remain in contact with the wound for a protracted period of time (such as bandages, sticking plasters, or other dressings) may be formulated such that the therapeutically effective amount of a compound promoting oestrogenic activity is administered relatively slowly. In contrast, medicaments that are intended only to be placed briefly in contact with the wound (such as swabs that will generally be used to “wipe down” a wound) may be formulated such that the therapeutically effective amount of the compound that promotes oestrogenic activity is administered in the relatively short time in which the medicament is in contact with the wound.

Suitable formulations may be selected with reference to how readily the compound that promotes oestrogenic activity is liberated from the solid or semi-solid medicament. For instance, in the case of a bandage, or the like, in which it is desired that release of the compound that promotes oestrogenic activity may be relatively slow, the compound that promotes oestrogenic activity may be incorporated in a matrix in which the interstices are sized such that the compound is released slowly to the wound. Alternatively, or additionally, the compound that promotes oestrogenic activity may be incorporated in a formulation from which it will be slowly released into the wound (in response to wound moisture, acidity, enzyme activity, or the like), as considered elsewhere in the specification.

By contrast, in the case of a swab, or the like, in which it is desired that release of the active compound is relatively rapid, it may be desired that the compound which promotes oestrogenic activity be provided in a liquid carrier that may be released rapidly from the substrate thereby providing the compound rapidly to the wound. In such uses the compound that promotes oestrogenic activity may be formulated in such a way that it is readily “accessible” to the wound (for example, dissolved in a suitable solute without the presence of binding partners or other agents that will form complexes with the oestrogenic compound).

Compounds that promote oestrogenic activity for use in medicaments of the invention may be provided as solid or liquid formulations. In either solid or liquid formulations the active compound will be incorporated in a carrier, from which the active compound will be released to a wound in order to exert its therapeutic activity.

Solid formulations for use in medicaments of the invention may include excipients such as binders. Suitable binders are well known to those skilled in the art.

Solid formulations for use in medicaments of the invention may, or may not, be contained in a containment membrane or coating, microspheres, microgranules or microcapsules. The materials for such containment membranes or coatings may be selected from any of a variety of biodegradable natural or synthetic materials. Suitable materials may provide resistance to diffusion of the active compound.

It may be preferred that suitable materials for use in containment membranes or coatings be selected such that they allow sustained release of the active compound to the wound. Examples of techniques by which this may be achieved will be well known to those skilled in the art, and will include the use of alternating layers of a suitable containment membrane or coating with layers of a carrier incorporating the active compound.

Suitable materials for use in containment membranes or coatings will generally degrade or be broken down over a period of time, thereby exposing the carrier, and allowing therapeutic release of the active compound from the carrier to the wound. The degradation or breakdown of suitable containment membranes or coatings may be caused by prolonged exposure to a wound. Factors that may mediate the degradation of such containment membranes or coatings will generally be the same as those that may cause the release of the active compound from the carrier. These are considered in more detail below, but it will be appreciated that the degradation or breakdown of containment membranes or coatings may typically be caused by moisture associated with the wound, or by the activity of enzymes active during wound healing.

Suitable solid formulations that may be used in medicaments of the invention may, for example, be selected from: powders; sprays, crystals; microneedles; solid compositions comprising microparticles, nanoparticles or liposomes; biomaterials; stents; films; artificial skin substances and wound dressings

Suitable liquid formulations that may be used in medicaments of the invention may, for example, be selected from: gels: thermosetting gels; creams; ointments; sprays; injectable solutions; irrigation solutions; other solutions of oestrogenic compounds; and liquid compositions comprising microparticles, nanoparticles or liposomes.

In the case where the medicament of the invention is a wound dressing, suitable solid formulations may be applied throughout the dressing, and particularly to the surface of the dressing that is to be placed into contact with the wound. Solid formulations may be applied as a coating that may be applied to all the material of the dressing, or may be applied to discrete portions of the dressing (for instance the surface of the dressing that is to be placed in contact with the wound). Solid formulations may also be provided as granules, microgranules, microparticles, nanoparticles or liposomes adhered to the material of the dressing. It will be appreciated that solid formulations may be provided in any such suitable form, so long as the active compound is able to be released from the solid formulation into the connective tissue underlying a wound site so that it can exert a therapeutic effect.

It will be appreciated that one of the advantages of applying solid formulations incorporating compounds that promote oestrogenic activity to wound dressings in this manner is that the release of the active compound to the wound, in order to provide a therapeutic amount of the active compound, occurs from the carrier, rather than from the material of the wound dressing itself. Thus the release characteristics of the carrier may be selected to achieve the optimal therapeutic effect of the active compound, while the physical properties of the material making up the wound dressing can be independently selected with a view to providing maximum effectiveness in term of compression, absorbency, or the like. In such cases it may generally be preferred that the material making up the wound dressing be one that is relatively “inert”, that is to say a material that does not in itself exert an effect on cells in the wound (save for any effects that may arise as a result of the material's structure, it's ability to absorb liquid from the wound, it's ability to exert compression on the wound, or the like).

Thus, merely by way of example, a sponge dressing material having pore/cell size that is well suited to drawing excess exudate from a wound, but which is of a material that is not well adapted for the release of active compounds, may be provided with a coating of a solid formulation that incorporates an active compound in a carrier that has characteristics optimised for the release of therapeutic amounts of the compound. In this way the beneficial effects of the material of the dressing may be combined with the beneficial characteristics of the chosen carrier.

The pore sizes of foams or sponges for use in medicaments of the invention may be such that they prevent substantial ingress of cells contributing to the wound healing process (e.g. keratinocytes associated with re-epithelialisation, or cells involved in granulation tissue formation) into the dressing. Foams or sponges to be used in medicaments of the invention may also be subject to surface treatments designed to reduce their adhesion to wounds to which they are applied. This may be advantageous in reducing damage that may otherwise be associated with changing dressings applied to wounds.

Foam or sponge pore sizes may be selected such that they facilitate the removal of exudates from the wound area, thus preventing or reducing maceration of the wound. The removal of exudates may be facilitated by the application of negative pressure to treated wounds, or to wound dressings applied to such wounds, and this may have further beneficial effects on the healing response.

Typically, release of the active compound to a wound may be brought about by the dissolution of the carrier in which the active compound is incorporated in moisture associated with (e.g. released from) the wound. In such cases it will be appreciated that suitable carriers may include water soluble compositions.

Other approaches may also be taken to control the release of therapeutic amounts of an active compound to a wound. For example, release of the active compound may be mediated by enzyme activity present in the wound. It is known that various proteloytic enzymes, such as plasmin and the Matrix Metalloproteinases (MMPs) are released during the process of wound healing. The natural proteloytic activity of such enzymes may be used to control release of the active compound to the wound. In this case, the carrier in which the active compound is incorporated may be one which is subject to proteloytic degradation by enzymes released during the wound healing response. For example, in the case of release mediated by the proteloytic enzyme MMP-1, the active compound may be incorporated in a carrier comprising a collagen matrix, wherein degradation of the collagen by MMP-1 will cause release of the active compound. Other examples of genes encoding proteolytic enzymes whose expression is significantly upregulated one day post-wounding, and which may therefore be used to control release of active compound into a wound the healing of which is to be accelerated, include cathepsin C, cathepsin L and MMP-9.

It has been suggested that the pH of wounds is lower than that of unwounded skin, and this observation may be considered when devising medicaments of the invention. Accordingly, carriers suitable for use in medicaments of the present invention may be ones in which the decreased pH of the wound to be treated contributes to therapeutic release of the active compound. Suitable materials that may be used in carriers in accordance with this embodiment of the invention will be well known to those skilled in the art.

An example of a pH-controlled carrier that releases a bound active compound in response to low pH can be found in a review by Gillies et al. (Pure Appl. Chem., 2004, 76, 1295-1307). Bae and co-workers (J. Controlled Release, 2003, 90, 363-374; ibid. 2003, 91, 103-113) prepared pH-sensitive micelles from poly(L-histidine)-block-PEO[PEO-b-P(His)], shown below. The micelles were prepared at pH 8, but were destabilised below pH 7.4 as evidenced by light transmittance measurements, light scattering, and fluorescent probe techniques. The triggering pH could be adjusted within the range of 7.2-6.6 by incorporation of different amounts of poly(L-lactic acid)-block-PEO(PLLA-b-PEO).

Release of an active compound (in this experimental case the anti-cancer drug doxorubicin) could be modulated using pH, and it was found that in vitro activity of the doxorubicin-loaded mixed micelles was heavily dependant on concentration.

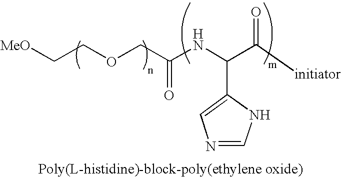

Although the passages above consider the benefits that may be provided when using different substances as a wound dressing material and as a carrier for the active compound, it will be appreciated that there may be circumstances in which it is desired that the material of a wound dressing serve as the carrier.

The use, in a medicament, of the same substance as both the material of a wound dressing and also as a carrier for an active compound may provide advantages in terms of reduced costs in the manufacture of such a medicament. It will be appreciated that medicaments in accordance with this embodiment of the invention may either be manufactured directly from the selected material already comprising the active compound, or may be manufactured before being treated such that they contain the active compound.

An example of the former suggestion may be the case in which a material (such as a hydrogel, collagen gel or other suitable polymer) containing an active agent in accordance with the invention is shaped or otherwise treated to provide a wound dressing (or part thereof). An example of the latter suggestion may be the case in which a material (such as an absorbent wound dressing) is treated with a solution containing the active compound, and the solution allowed to at least partially evaporate, thereby leaving a substantially dry wound dressing permeated with the active compound. The active compound may then be released from the dressing material by dissolution in fluids released from the wound.

Medicaments of the invention may be formulated in the form of compositions comprising the active compound, which promotes oestrogenic activity, in a polymer matrix. Suitable polymer matrices may typically comprise materials selected from the group consisting of: acrylic copolymers; vinyl acetate; natural or synthetic rubbers;

The skilled person will be readily able to identify polymer compositions that may be used in the manufacture of medicaments of the invention from those known in the prior art. Specific examples of formulations comprising polymer matrices suitable for use in medicaments of the present invention are described in greater detail below.

A suitable matrix that may be used for transdermal delivery of active compounds in medicaments of the present invention is described in U.S. Pat. No. 5,252,334 and in U.S. Pat. No. 5,770,219. The disclosure of these documents, particularly in their description of the manufacture of such matrices, is incorporated by reference.

The matrix described in these documents is formed of a skin-adhesive acrylate copolymer, and attains rates of delivery of the active compound suitable to provide therapeutically effective amounts thereof to a wound. For example, the matrices described in U.S. Pat. No. 5,252,334 can be used to administer approximately 0.2 μg of 17β-oestradiol per hour per square centimetre of skin. This rate of delivery described may be achieved without the addition of drug delivery rate enhancers. The skilled person will immediately appreciate that rates of administration of an active compound, for example to provide a dose of up to 300 ng of 17β-oestradiol per centimetre of skin over a 24 to 48 hour period, may be readily obtained by altering the amount of the active compound incorporated in the matrix, as appropriate.

Alternatively or additionally, the matrices and formulations described in U.S. Pat. No. 5,252,334 may be applied to a wound (or site where a wound is to be formed) for a period of time sufficient to allow a therapeutically effective amount of an active compound to be administered to a wound. It will be appreciated that a suitable period of administration may be in the region of an hour or less (for example half an hour), and that exposure to a medicament of the invention for such relatively short periods of time may be undertaken as part of the immediate care (such as post-operative care) associated with surgical interventions or grafting procedures. The relatively short time in which medicaments in accordance with this embodiment of the invention may release a therapeutically effective amount of an active compound to a wound, or a site where a wound is to be formed, is particularly advantageous in the context of surgical procedures (such as grafting procedures) since the medicament need only be applied relatively briefly (for example in the time during which a patient is being prepared for surgery) to allow effective acceleration of healing to be promoted.

A suitable medicament comprising an active compound incorporated in a matrix of the type described in U.S. Pat. No. 5,252,334 may consist of a first layer laminated to a second layer, where the first layer effective acts as a backing sheet (made of a material which is substantially impermeable to the active compound) and the second layer comprises an adhesive matrix in which the active compound is incorporated (the adhesive matrix comprising a copolymer of 2-ethylhexyl acrylate and at least one co-monomer selected from the group consisting of vinyl acetate, acrylic acid, and methyl acrylate). The adhesive matrix thus secures the medicament to the site to be treated, and also administers a therapeutically effective amount of the active compound. The backing sheet can function to protect the matrix containing the active compound, as well as shielding external materials from the adhesive layer, and preventing unwanted release of the active compound to tissues other than those to be treated.

U.S. Pat. No. 4,814,168 describes “dermal compositions” that may be used as a suitable matrix by which active compounds of the invention may be administered.

The compositions of U.S. Pat. No. 4,814,168 comprise an active ingredient incorporated in a multi-polymer of vinyl acetate, polyethylene and optionally one or more monomers, a natural or synthetic rubber and a tackifying agent. The ratio of the multipolymer to the rubber is, respectively, about 1:1 to about 10:1, although this may more preferably be between 1:1 and 5:1, and even more preferably 3:1. The multi-polymer can be a copolymer, or terpolymer also including an acrylic and/or methacrylic acid monomer. The composition can additionally contain or employ other ingredients known for use in pressure sensitive adhesives including crosslinking agents, plasticizers, fillers and anti-oxidants.

Dermal compositions of this type suitable for use as matrices from which therapeutically effective amounts of active compounds may be administered in accordance with this embodiment of the invention can optionally contain a crosslinking agent, tackifiers, penetration enhancers and other ingredients known for use in adhesives for the transdermal delivery of drugs.

The dermal compositions can be produced by a variety of methods known to those skilled in the art. By way of example, these may include the homogenous mixing of the active compound multi-polymer, and optional crosslinking agent and additional ingredients in aqueous solution followed by removal of excess water. The composition is prepared by mixing the active compound and an essentially non-tacky polymer, (namely the multi-polymer) with an elastomer (namely the rubber) and a tackifying agent. The composition maintains its adhesive properties even where the active acts as a plasticizer or solvent. The tackifying agent increases tack and adhesiveness.

Although the structure of the compositions described in U.S. Pat. No. 4,814,168 has not been analyzed, it is suggested that the two polymers incorporated in the compositions result in a heterogeneous mix, the elastomer performing as an interpenetrating polymeric network in the multi-polymer.

A further example of a suitable formulation by which active compounds may be administered to a wound (or site to be wounded) comprises micellar nanoparticles incorporating the active agent. Suitable micellar nanoparticles may be made by hydrating a mixture of an oil, a stabilizer/surfactant, and an alcoholic initiator (normally ethanol or methanol) with an aqueous solution comprising the active compound. A method by which micellar nanoparticles may be produced is described fully in U.S. Pat. No. 5,629,021. The methods of manufacturing micellar nanoparticles comprising active compounds described in that disclosure are incorporated by reference. The micellar nanoparticles that may be produced using the methods described in U.S. Pat. No. 5,629,021 are normally less than 100 nanometers in diameter.

The use of micellar nanoparticles confers a number of advantages in the context of the present invention. One specific advantage of such materials lies in their delivery of natural or synthetic hormones, such as 17β-oestradiol, that will frequently be used as active compounds in the medicaments or methods of the invention. These materials often have solubility problems in that they are often only soluble in materials such as ethanol. It is known that ethanolic solutions can be difficult to incorporate in stable particulate systems. The inventors have found that ethanol represents a preferred solvent for use in the medicaments of the invention. Micellar nanoparticles offer a notable advantage in that they allow materials that are soluble in any solute selected from water, oil, or an initiator (i.e., ethanol or methanol) to be incorporated into stable particles with mean diameters between about 30 and 1000 nanometers.

The small size of micellar nanoparticles marks them apart from many other compositions that may be used for the administration of therapeutic amounts of compounds that promote oestrogenic activity, and also lends itself to certain of their applications. The skilled person will recognise that other synthetic particles (such as liposomes, nonphospholipid lipid vesicles and microcapsules) are normally a micron, or larger, in size. In contrast, micellar nanoparticles may have mean diameters between about 30 and 1000 nanometers, and will frequently have sizes less than 100 nanometers diameter. Most preparations have particle diameters between 30 to 500 nanometers, are mixable in water, and filterable through either 0.2 or 0.45 micron filters.

Another notable advantage of micellar nanoparticles lies in their improved delivery of active compounds (such as 17β-oestradiol) to a wound. Delivery of the active compound, even through intact skin (as in cases of prophylactic treatment prior to wounding) is facilitated, since the small size of the micellar nanoparticles allows their easy penetration through the epidermis and into the dermis/connective tissue where they may exert their therapeutic effect. Micellar nanoparticles comprising 17β-oestradiol have been shown to be effective in the topical administration of that compound to the skin.

Finally, micellar nanoparticles can be stored stably for protracted periods at temperatures of between −20 and 25° C. This provides clear advantages in cases where it is wished to produce medicaments of the invention that will have relatively long “shelf-lives” between manufacture and use.

It is known that certain agents can increase the ability of active compounds of the invention to cross the skin. These agents are typically referred to as “permeation enhancers”, terminology that will be adopted in the present disclosure.

The need for permeation enhancers may arise since the intact skin provides an effective barrier to the uptake of active compounds that may be used in accordance with the invention. Thus it will be appreciated that permeation enhancers are most likely to be of benefit in medicaments or methods that are to be utilised prophylactically (discussed elsewhere, in which medicaments or methods of the invention are administered prior to the formation of a wound in order to “prime” the treated area so a wound formed at that site will benefit from accelerated healing as soon as the wound is formed), since in these cases the active compounds of the medicaments will have to pass through the unwounded skin in order to reach the underlying connective tissue, where the inventors believe that their therapeutic effects are mediated.

In the case of medicaments administered to sites of existing wounds, the wound (whether full thickness or partial thickness) will generally impair the skin's barrier function. As a result, this will normally facilitate the uptake of therapeutically effective amounts of the active compounds from medicaments of the invention without the need for permeation enhancers.

Although, it may not generally be necessary to incorporate permeation enhancers in medicaments of the invention for use at sites of existing wounds, there may be circumstances in which it will be desirable to use such agents, for instance to hasten uptake of the active compound from the medicament.

Preferably the acceleration of wound healing using the medicaments or method of the invention may give rise to a healing time 1 day, 2 days, or 3 days faster than that occurring in a control-treated or untreated wound. Healing time may be calculated as the time elapsing between formation of a wound and complete re-epithelialisation of the wound. More preferably accelerated healing in accordance with the invention may give rise to a time to a healing time that is at least 4 days, 5 days or 6 days faster than that occurring in a control-treated or untreated wound. It is even more preferred that accelerated healing may give rise to a time to a healing time that is at least 7 days, 8 days or 9 days faster than that occurring in a control-treated or untreated wound, and most preferably accelerated healing may give rise to a time to re-epithelialise that is at least 10 days or greater than that occurring in a control-treated or untreated epithelium.

With respect to reducing the time that must elapse to allow re-harvesting of graft material from skin donor sites, preferably the acceleration of healing may give rise to a time to re-harvesting that is 1 day, 2 days, or 3 days faster than that occurring in a control-treated or untreated graft donor sites. More preferably acceleration of the healing of wounds using the medicaments and methods of the invention may give rise to a time to re-harvesting that is at least 4 days, 5 days or 6 days faster than that occurring in a control-treated or untreated graft donor site. It is even more preferred that accelerated healing of wounds may give rise to a time to re-harvesting that is at least 7 days, 8 days or 9 days faster than that occurring in a control-treated or untreated graft donor site, and most preferably accelerated healing of wounds may give rise to a time to re-harvesting that is at least 10 days or greater than that occurring in a control-treated or untreated graft donor site.

The acceleration of wound healing that may be achieved at graft donor sites, or other sites of partial thickness wounds (such as those associated with skin peels or dermabrasion), may help to reduce redness of such sites, and also to reduce moisture loss that may otherwise occur through such wounds.

The inventors have found that the medicaments and methods of the invention are able to accelerate healing of wounds when used either prior to the formation of a wound, or when used after a wound has already been formed. The use of the medicaments and methods of the invention prior to formation of a wound (in which case it is believed that the action of the medicament or method is to “prime” the site where wounding will occur so that the healing response is accelerated immediately upon formation of the wound) is referred to as “prophylactic use” for the purposes of the present disclosure.

The prophylactic use of agents in accordance with the invention to accelerate the healing of wounds is a preferred mode of use in accordance with the invention. It will be appreciated that such use is most suitable in the case where the time and location of prospective wound formation is known, and may be particularly suitable for use in acceleration of wounds associated with grafting procedures. The medicaments or methods of the invention may be used both prophylactically (i.e. before a wound is formed) and after wound formation. This approach, with the medicament used both before and after wounding, represents a preferred embodiment of the invention.

Experiments undertaken by Mahmoud and co-workers using a reconstructed epidermis model to assess uptake through the skin (i.e. transdermally) indicate that application of 100 μg of oestradiol in a gel to an epidermal surface leads to 15.7 μg of the applied oestradiol being found in the skin or having passed through the skin in six hours from application. Accordingly, the amounts of oestradiol that should be administered to an epidermal surface in order to obtain uptake of a therapeutically effective amount of up to 300 ng of oestradiol may be readily determined by those skilled in the art. A gel capable of providing a therapeutically effective amount of an active agent through the epidermis (for instance, at a site where a wound may be formed) thus represents a preferred medicament of the invention.

Preferably the acceleration of healing using the medicaments or method of the invention may give rise to a healing time 1 day, 2 days, or 3 days faster than that occurring in a control-treated or untreated wound. Healing time may be calculated as the time elapsing between formation of a wound and complete re-epithelialisation of the wound (indicated by complete epithelial coverage of the wound). More preferably the acceleration of wound healing in accordance with the invention may give rise to a time to a healing time that is at least 4 days, 5 days or 6 days faster than that occurring in a control-treated or untreated wound. It is even more preferred that acceleration of wound healing may give rise to a healing time that is at least 7 days, 8 days or 9 days faster than that occurring in a control-treated or untreated wound, and most preferably acceleration of wound healing may give rise to a time to re-epithelialisation that is at least 10 days or greater than that occurring in a control-treated or untreated wound.

The inventors have found that the medicaments and methods of the invention are able to promote re-epithelialisation, and hence accelerated wound healing, when used either prior to the formation of a wound, or when used after a wound has already been formed. The use of the medicaments and methods of the invention prior to formation of a wound (in which case it is believed that the action of the medicament or method is to “prime” the site where wounding will occur so that healing is accelerated immediately upon formation of the wound) is referred to as “prophylactic use” for the purposes of the present disclosure.

The prophylactic use of agents in accordance with the invention to accelerate the healing of wounds is a preferred mode of use in accordance with the invention. It will be appreciated that such use is most suitable in the case where the time and location of prospective wound formation is known, and may be particularly suitable for accelerating the healing of wounds associated with surgical procedures. However, prophylactic use of the medicaments or methods of the invention may also be of use in situations where there is an increased likelihood of wounding occurring. The inventors have found that administration of agents in accordance with the invention immediately prior to formation of a wound (e.g. in the hour preceding wounding, or preferably in the forty minutes or thirty minutes preceding wounding, and more preferably in the ten minutes preceding wounding) is highly effective, though administration at earlier times (e.g. up to 24 or 48 hours before wounding) may also beneficially accelerate the healing of wounds. The prophylactic use of methods and medicaments of the invention is a preferred embodiment of the invention, and is particularly preferred for accelerating the healing of skin graft donor and/or recipient sites.

Injection, and particularly intradermal injection, constitutes a preferred manner in which the medicaments of the invention may be administered (or the methods of the invention effected), as considered elsewhere in the specification. In the case of prophylactic use, it may be particularly preferred that a medicament of the invention be administered by intradermal injection to a site where wounding will take place. If the medicament is administered only a short time prior to wound, then intradermal injection of this type will typically lead to the formation of a raised bleb which will remain at the time of wounding. A wound may then be formed through the bleb. Wounds formed in this way will benefit from accelerated healing in accordance with the present invention. Alternatively, blebs formed by intradermal injection of medicaments of the invention may be allowed to resolve before a wound is formed.

The medicaments and methods of the invention may also be used to accelerate the healing of wounds once the wound in question has already been formed. This use will be the use generally adopted in respect of many wounds, including accidental wounds and those associated with skin peel procedures (as opposed to prophylactic treatment, which may be preferred in the case of wounds associated with surgical procedures).

In the event that the medicaments or methods of the invention are to be used to accelerate the healing of an existing wound, it is preferred that such use should occur as early as possible after formation of the wound. That said, the medicaments or methods of the invention may help to accelerate healing of a wound if used at any time up until full healing has occurred (for example even if administered to a partially healed wound the medicaments of the invention may accelerate the healing in respect of that portion of the wound that remains as yet unhealed).

Factors that may be considered in relation to the “window” in which medicaments or methods of the invention may be beneficially employed such that they are able to accelerate the healing of wounds will include: the nature of the wound in question (for example: is the wound at a site that is generally subject to “fast” or “slow” healing?); the severity of the wound (what is the extent of the damage that has occurred?); and the size of the damaged area. Thus in the case of a wound of large area, or in a site that is naturally associated with slower than average healing, the methods or medicaments of the invention may be still be effective to accelerate the healing of the wound even if administered relatively late in the healing response. Thus, although the medicaments or methods of the invention may preferably be administered within the first one to 24 hours after formation of an acute wound, beneficial acceleration of healing may also be brought about if administered up to ten, or more, days after the wound is formed.

It will be appreciated that in the case of chronic wounds, the period in which the medicaments or methods of the invention may be beneficially employed will be considerably longer. Chronic wounds may persist for many years, and the healing of wounds that may be many years old may be beneficially accelerated using the medicaments or methods of the invention.

Acceleration of the healing of wounds may be achieved using only a single administration of the medicaments or methods of the invention. Due to the simplicity of this therapeutic regime it constitutes a preferred use of the medicaments and methods of the invention.

However, there may be cases in which it is preferred that the medicaments or methods of the invention be used in repeated incidences of therapy. Thus treatment to accelerate healing of a wound may involve administration of medicaments of the invention on more than once occasion. Use in this manner may be preferred in the case of large wounds, or of wounds that are resistant to treatment, or subject to retarded healing (such as chronic wounds). Generally medicaments of the invention may be administered to a wound as required until healing has been achieved. By way of example, medicaments of the invention may be administered daily (or on multiple occasions within a given day), or may be administered after a delay of multiple days.

Generally when the medicaments or methods of the invention are to be used in multiple therapeutic incidences administration should be repeated until acceleration of healing has been achieved to a clinician's satisfaction.

Although the medicaments or methods of the invention may be used on a single instance (either before or after formation of a wound), it may be preferred that the medicaments or methods of the invention are utilised both before and after wounding.

It may be preferred that the medicaments of the invention are administered to a site where they are to have their effect around the time of wounding, or immediately prior to the forming of a wound (for example in the period up to six hours before wounding, and particularly in the period of 10, 20, 40 or 60 minutes prior to wound formation) or the medicaments may be administered at an earlier time before wounding (for example up to 48 hours before a wound is formed). The skilled person will appreciate that the most preferred times of administration prior to formation of a wound will be determined with reference to a number of factors, including the formulation and route of administration of the selected medicament, the dosage of the medicament to be administered, the size and nature of the wound to be formed, and the biological status of the patient (which may determined with reference to factors such as the patient's age, health, and predisposition to healing complications or adverse scarring) and the half-life of the compound that promotes oestrogenic activity in the body.

It may be particularly preferred that the methods or medicaments of the invention may be administered both before and after formation of a wound. The inventors believe that administration of the medicaments of the invention immediately prior to the formation of a wound, followed by administration of a compound that promotes oestrogenic activity for one or more days following wounding, is particularly effective in accelerating healing.

For the purposes of the present specification, an “agent” or “agent of the invention” will be a therapeutically effective compound able to promote oestrogenic activity. It will be appreciated that all such suitable agents may be incorporated in medicaments in accordance with the invention, and all may be used in the methods or uses of the invention. The medicaments of the invention represent preferred compositions by which a therapeutically effective amount of a compound that promotes oestrogenic activity may be administered in order to put the methods of the invention into practice.

17β-oestradiol is a preferred example of an agent, or agent of the invention, as considered elsewhere in the specification.

It will be appreciated that the amount of a medicament of the invention that should be provided to a wound, in order that a therapeutically effective amount of compound that promotes oestrogenic activity may be administered, depends on a number of factors. A number of these are discussed elsewhere in the specification, and include the biological activity and bioavailability of the agent present in the medicament, which in turn depends, among other factors, on the nature of the agent and the mode of administration of the medicament. Other factors in determining a suitable therapeutic amount of a medicament may include:
A) The half-life of the agent in the subject being treated.
B) The specific wound to be treated (e.g. accelerating healing of an acute wound or a chronic wound).
C) The age of the subject.
D) The size of the site to be treated.

The frequency of administration will also be influenced by the above-mentioned factors and particularly the half-life of the chosen agent within the subject being treated.

Frequency of administration will depend upon the biological half-life of the agent used. Typically a cream or ointment containing an agent of the invention should be administered to a target tissue such that the concentration of the agent at a wound is maintained at a level suitable to accelerate wound healing. This may be achieved by a single administration of a composition incorporating a compound that promotes oestrogenic activity, or may require administration of such a composition daily or even several times daily.

Medicaments of the invention, may be administered by any suitable route capable of achieving the desired effect of accelerating wound healing, but it is preferred that the medicaments be administered locally at a wound site or site where a wound is to be formed.

Administration (and particularly topical administration) of the medicaments of the invention may be effected as part of the initial and/or follow up care for the wounded area.

As suggested elsewhere in the specification, the agents of the invention may be provided on a dressing or patch, which may be used to cover a wound the healing of which is to be accelerated. It will be appreciated that such a dressing or patch used to administer an agent of the invention may preferably be provided in a sterile form.

The agents of the invention may be released from a device or implant, or may be used to coat such a device e.g. a stent or controlled release device.

It will be appreciated that the vehicle of a composition comprising agents of the invention should be one that is well tolerated by the patient and allows release of the agent to the wound to which the composition is provided. Such a vehicle is preferably biodegradable, biocompatible, bioresolveable, bioresorbable and/or non-inflammatory. If the composition is to be applied to an existing wound then the pharmaceutically acceptable vehicle will be one that is relatively “mild”.

Delayed release devices may be particularly useful for patients requiring protracted treatment with the medicaments or methods of the invention, such as those where it is wished to accelerate healing of chronic wounds. Delayed release devices may be particularly advantageous when used for the administration of an agent that would otherwise normally require frequent administration (e.g. at least daily administration by other routes).

Daily doses of an agent of the invention may be given as a single administration (e.g. a daily application of a topical formulation or a daily injection). Alternatively, the agent of the invention may require administration twice or more times during a day. Each such administration may provide a therapeutically effective amount of the agent, or a known fraction of such a therapeutically effective amount. In a further alternative, a slow release device may be used to provide optimal doses of an agent of the invention to a patient without the need to administer repeated doses.

A dose of a composition comprising agents of the invention may preferably be sufficient to provide a therapeutically effective amount of compound that promotes oestrogenic activity in a single administration. However, it will be appreciated that each dose need not in itself provide a therapeutically effective amount of a compound that promotes oestrogenic activity, but that a therapeutically effective amount may instead be built up through repeated administration of suitable doses.

Various suitable forms are known for compositions comprising agents of the invention. In one embodiment a pharmaceutical vehicle for administration of an agent of the invention may be a liquid and a suitable pharmaceutical composition would be in the form of a solution. Such a solution may be administered by injection, or by other routes, such as by a spray. An agent of the invention may be formulated as part of a cream, gel or ointment. In a further embodiment the agent of the invention may be formulated as a part of a pharmaceutically acceptable patch providing delivery of a therapeutically effective compound that promotes oestrogenic activity to a wound or a site where a wound is to be formed.

Liquid vehicles may be used in preparing solutions, suspensions, emulsions, syrups, elixirs and pressurized compositions (sprays). The agent of the invention can be dissolved or suspended in a pharmaceutically acceptable liquid vehicle such as water, an organic solvent, a mixture of both or pharmaceutically acceptable oils or fats. It may generally be preferred that the medicaments or methods of the invention make use of oestrogenic compounds diluted in an aqueous solution (i.e. a solution in which water is the major diluent). It may be preferred that medicaments or methods of the invention make use of oestrogenic compounds dissolved in diluents other than oils. In the case where it is desired to utilise an oil as a diluent in accordance with a medicament or method of the invention it may generally be preferred to use synthetic oils or mineral oils.

Suitable diluents, whether aqueous or otherwise, may preferably be substantially free from allergens or toxins, such as those that may be found in naturally occurring products (such as nut or vegetable oils) that may otherwise be used as diluents in medicaments or methods of the invention. It will be appreciated that the considerations above are of particular concern in the case of medicaments or methods of the invention in which oestrogenic compounds are administered by means of injection. Furthermore, the inventors have found that the use of oil-based diluents in the preparation of compositions for administration by localised injection (such as intradermal injection) to wounds, or sites where wounds are to be formed, may generally be disadvantageous, and can give rise to deleterious effects that may retard the wound healing process and lead to the production of abnormally wide wounds. Thus it may be preferred not to use oils in the preparation of injectable medicaments in accordance with the invention.

The liquid vehicle can contain other suitable pharmaceutical additives such as solubilizers, emulsifiers, buffers, preservatives, sweeteners, flavouring agents, suspending agents, thickening agents, colours, viscosity regulators, stabilizers or osmo-regulators. Suitable examples of liquid vehicles for parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). A solution comprising 95% phosphate buffered saline (PBS) and 5% ethanol is a particularly preferred vehicle for use in compositions comprising 17β-oestradiol as the agent of the invention. For parenteral administration, the vehicle can be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellant.

Liquid pharmaceutical compositions which are sterile solutions or suspensions can be utilized by, for example, intramuscular, intraperitoneal, intradermal, intrastromal (cornea), intraadventitial (blood vessels) or subcutaneous injection. Sterile solutions can also be administered intravenously. The agent of the invention may be prepared as a sterile solid composition that may be dissolved or suspended at the time of administration using sterile water, saline, or other appropriate sterile injectable medium (such as PBS). Vehicles are intended to include necessary and inert binders, suspending agents, lubricants and preservatives.

Agents of the invention may be used to accelerate healing of “internal” wounds (i.e. wounds occurring within the body, rather than on an external surface of the body). Examples of internal wounds include penetrative wounds that pass through the skin into underlying tissues, and wounds associated with surgical procedures conducted within the body.

It will be appreciated that the use of medicaments or methods of the invention to accelerate the healing of internal wounds will necessitate the use of suitable routes of administration, thereby requiring the formulation of the agents of the invention in a manner that allows their delivery to the wound in question. For example, medicaments in accordance with the invention for accelerating the healing of wounds in the lungs or other respiratory tissues may be formulated for inhalation. In another preferred embodiment, medicaments in accordance with the invention for accelerating the healing of wounds in the body cavities (such as the abdomen or pelvis) may be formulated as a lavage, gel or instillate.

Known procedures, such as those conventionally employed by the pharmaceutical industry (e.g. in vivo experimentation, clinical trials etc), may be used to establish specific formulations of compositions comprising agents of the invention and precise therapeutic regimes for administration of such compositions (such as daily doses of the active agent and the frequency of administration).

The inventors believe that the amount of a compound that promotes oestrogenic activity that may be administered to a wound (or site where a wound is to be formed) in a single incidence of treatment, for example by means of a topical injection, may be up to 1.1 nmoles/cm² of area administered.

By way of further example, the preferred amount of compound that promotes oestrogenic activity to be administered to the connective tissue/dermis of a wound over a period of approximately 24 hours may be in the region of 0.36 nmoles to 0.72 nmoles per cm² of wound or dermis (if administered by injection).

The skilled person will appreciate that the suggestions above are provided for guidance. In particular it will be appreciated that the amount of a compound that promotes oestrogenic activity to be administered via topical administration may be altered depending on permeability of the tissue or organ to which the topical composition is administered. Thus in the case of relatively impermeable tissues or organs it may be preferred to increase the amount of the agent administered. Such an increased amount of a compound that promotes oestrogenic activity may still represent a therapeutically effective amount, if the amount of the agent taken up into the tissue or organ where wound healing is to be accelerated is therapeutically effective (i.e. if a therapeutically effective amount permeates the tissue or organ where healing is to be accelerated, irrespective of the fact that a larger amount of the agent may remain on the surface of, and unable to penetrate, the tissue or organ being treated).

In a particularly preferred embodiment, the compound that promotes oestrogenic activity (for instance 17β-oestradiol) may be administered as a 0.7 μM to 11.0 μM solution, with 100 μL of such a solution administered per cm² of area administered (particularly per centimetre of wound) over a 24 hour period.

The skilled person will appreciate that effective therapeutic amounts of a compound that promotes oestrogenic activity may be determined with reference to the concentration of the agent that is attained in the organ or tissue to which they are administered. The information regarding therapeutically effective dosages set out herein will provide sufficient guidance to allow the skilled person to calculate the local concentrations of a compound that promotes oestrogenic activity (such as 17β-oestradiol) established by intradermal injection, and, based on these values, to determine suitable amounts of such agents that may be administered by other routes in order to achieve equivalent local concentrations. It will be appreciated that the tissue concentration of a compound that promotes oestrogenic activity to be established should be one at which the selected compound achieves the therapeutically effective level of oestrogenic activity required.

The inventors have found that 17β-oestradiol may be administered by way of an injectable solution containing approximately 100 to 200 ng 17β-oestradiol per 100 μL solution in order to accelerate re-epithelialisation (and thus accelerate wound healing) when administered as an intradermal injection.

It will be appreciated that the guidance as to doses and amounts of agents that promote oestrogenic activity to be used provided above is applicable both to medicaments of the invention, and also to the methods of the invention.

Medicaments of the invention may be used to accelerate wound healing as a monotherapy (e.g. through use of medicaments of the invention alone). Alternatively the methods or medicaments of the invention may be used in combination with other compounds or treatments for the acceleration of re-epithelialisation, or other means by which healing of a wound may be accelerated. Suitable compounds that may be used as parts of such combination therapies will be well known to those skilled in the art.

Although it is preferred that the medicaments or methods of the invention be used in human patients, it will be appreciated that many of the advantages that may be gained as a result of accelerated healing of human wounds are also are also applicable to healing of wounds in other animals, particularly veterinary or domestic animals (e.g. horses, cattle, dogs, cats etc). Accordingly it will be recognised that the medicaments and methods of the invention may also be used to accelerate the healing of wounds of non-human animals.

The invention will now be further described with reference to the accompanying Experimental Results and Figures in which:

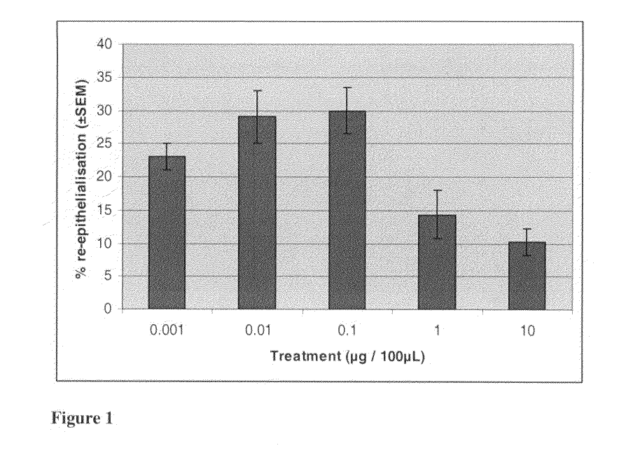

FIG. 1 shows a bar graph illustrating the results of a study comparing re-epithelialisation in rat excisional wounds treated with medicaments of the invention with re-epithelialisation in control wounds treated with amounts of 17β-oestradiol suggested in the prior art;

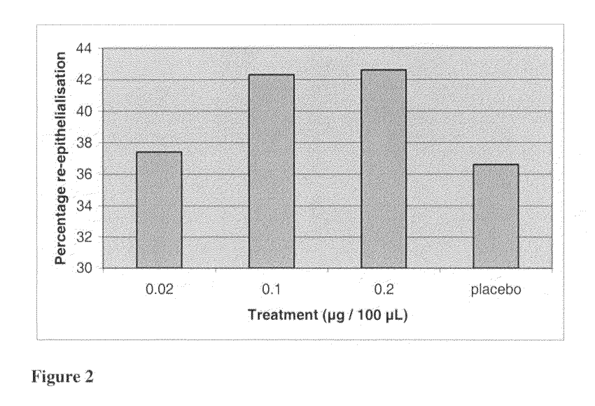

FIG. 2 compares re-epithelialisation in treated and control human punch biopsies; and

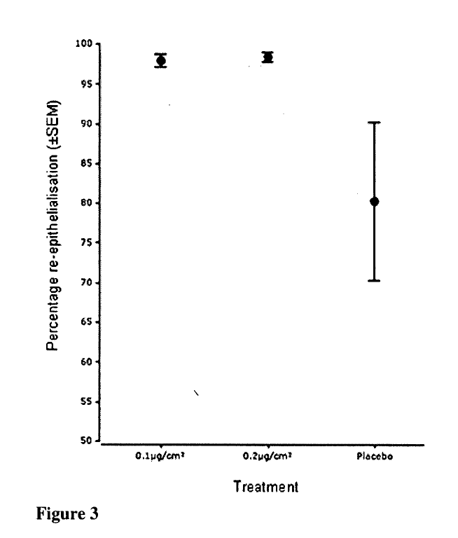

FIG. 3 compares re-epithelialisation in treated and control partial thickness wounds in pigs.

### EXPERIMENTAL RESULTS

### 1 Comparison of Medicaments of the Invention with Medicaments of the Prior Art

The following study was undertaken to compare acceleration of re-epithelialisation, and hence wound healing, brought about using the medicaments of the invention, with the level of healing brought about using medicaments incorporating the amounts of oestrogenic compounds disclosed in the prior art. The model of wound healing utilised made use of full thickness excisional wounds in experimental rats.

### 1.1 Materials and Methods

Experimental medicaments were prepared, using the following constituents:

| | |
|---|---|
| 17β oestradiol | 0.01 μg, 0.1 μg, 1 μg, 10 μg or 100 μg |
| Ethanol | 50 μl |
| KH2PO4 | 0.21 mg |
| NaCl | 9.0 mg |
| Na2HPO4•2H20 | 0.482 mg |
| 0.1M NaOH | q.s. to pH 7.2 |
| 0.1M HCl | q.s. to pH 7.2 |
| Water for injection | to 1 ml |

The experimental medicaments produced contained concentrations of 17β-oestradiol as follows:
i) 0.001 μg 17β-oestradiol/100 μl of medicament (0.000001%);
ii) 0.01 μg 17β-oestradiol/100 μl of medicament (0.00001%);
iii) 0.1 μg 17β-oestradiol/100 μl of medicament (0.0001%);
iv) 1 μg 17β-oestradiol/100 μl of medicament (0.001%); and
v) 10 μg 17β-oestradiol/100 μl of medicament (0.01%)

Of these, i) to iii) constituted a medicament of the invention, whereas iv) and v) contained amounts of the oestrogenic compound 17β-oestradiol shown to accelerate healing in the prior art (being respectively 0.001% and 0.01% solutions of 17β-oestradiol).

### 1.1.3 Wound Healing Model

A rat excisional wound model was used to investigate the acceleration of re-epithelialisation, and hence wound healing, brought about by the medicaments tested.

100 μl of the medicament to be tested was administered by intradermal injection at sites on the dorsal surface of experimental rats, 10 minutes prior to wounding. The amount administered was sufficient to pre-treat an area of 1 cm² prior to wounding. Following this pre-treatment a 5 mm full thickness punch biopsy was formed at the injected site (creating a wound with an initial area of 0.196 cm²). One injection and excisional wound was made per animal, and ten adult male Sprague-Dawley rats were used per treatment group.

### 1.1.4 Assessment of Acceleration of Wound Healing

Animals were killed, and their wounds harvested, three days after wounding. The wounds were excised and treated for histology. Histological sections from wounds treated with medicaments of the invention and from wounds treated with prior art medicaments were studied and the percentage of re-epithelialisation exhibited by the different wounds calculated and compared. All histological assessments of early wound healing events were made using preserved 5 micron-thickness wound sections taken from the widest part of each excised biopsy site. Sections were stained with Haematoxylin and Eosin to aid visualisation of structural features, and measurements made using image analysis software. The distance traveled by the epithelium is derived from two histological measurements: the freehand total wound diameter and the freehand non-epithelialised wound diameter. The non-epithelialised diameter is subtracted from the total wound diameter, to determine the distance which the new epithelium has covered since the time of wounding. Percentage re-epithelialisation for the new epithelium was calculated as a proportion of the total wound diameter. The average percentage re-epithelialisation value was calculated from the wounds of the ten animals within each treatment group.

### 1.2 Results

Wounds treated with the 1 μg/100 μL (0.001%) solution of 17β-oestradiol exhibited 14.63% re-epithelialisation as assessed using the protocol described above.

Wounds treated with the 10 μg/100 μL (0.01%) solution of 17β-oestradiol exhibited 10.30% re-epithelialisation as assessed using the protocol described above.

In contrast, wounds treated with the medicaments of the invention exhibited much increased re-epithelialisation assessed as described above. Wounds treated with the medicament providing 0.001 μg 17β-oestradiol per 100 μl (0.000001%) exhibited 23.02% re-epithelialisation, while those treated with the 0.01 μg/100 μl (0.00001%) medicament exhibited 29.03% re-epithelialisation, and those treated with the 0.1 μg/100 μl (0.0001%) medicament 29.95% re-epithelialisation.

Thus the results of this study (shown in FIG. 1) clearly indicate that the medicaments of the invention are able to accelerate wound healing (as demonstrated by an increase in the rate of re-epithelialisation) to a greater extent than medicaments described in the prior art, even though the medicaments of the invention incorporated a far lower concentration of 17β-oestradiol. It will be recognised that the ability to achieve greater acceleration of wound healing while using a smaller amount of a compound that promotes oestrogenic activity confers surprising advantages over the prior art.

### 2 Medicaments of the Invention Accelerate Healing of Full Thickness Cutaneous Excisional Wounds in Humans

The inventors studied the effects of medicaments of the invention in promoting an increase in the rate of re-epithelialisation (and hence healing) of human punch biopsy wounds.

### 2.1 Materials and Methods

### 2.1.1 Medicaments of the Invention

Medicaments of the invention were prepared using the constituents described above. Three concentrations of the medicaments were prepared, as follows:
i) 0.02 μg 17β-oestradiol/100 μl of medicament (0.00002%)
ii) 0.1 μg 17β-oestradiol/100 μl of medicament (0.0001%)
iii) 0.2 μg 17β-oestradiol/100 μl of medicament (0.0002%)

### 2.1.2 Wound Healing Model

Healthy male and post-menopausal female volunteers were chosen as the subjects of the study. 44 healthy subjects were recruited to the study, of whom, 40 were healthy males (aged 18-75 years) and 4 were post-menopausal females (aged 53-69 years). The average age of subjects recruited to this study was 42 years. The post-menopausal status of female subjects was confirmed by quantification of serum levels of oestradiol and follicle stimulating hormone (FSH). Serum levels of oestradiol were required to be <90 pmol/L, and follicle stimulating hormone levels >31 IU/L.

Each subject had three wounds treated with active drug, one corresponding to each of three concentrations of the medicaments produced; a fourth wound was treated with placebo control. The test medicaments and placebo control were each administered intradermally, as a single dose, at a volume of 100 μl per wound site (the upper, inner aspect of each arm), 10-minutes before wounding.

Subjects received 3 mm punch biopsies at the sites where the medicaments had been administered. After wounding, all sites received moist wound healing dressings (Standard Care).

### 2.1.3 Assessment of Acceleration of Wound Healing

Biopsy sites were excised, using a 5 mm punch biopsy, three days after wounding. Excised wounds were preserved for histological assessment of re-epithelialisation by image analysis. All histological assessments of early wound healing events were made using preserved 5 micron-thickness wound sections taken from the widest part of each excised biopsy site. Sections were stained with Haematoxylin and Eosin to aid visualisation of structural features, and measurements made using image analysis software. The distance traveled by the epithelium is derived from two histological measurements: the freehand total wound diameter and the freehand non-epithelialised wound diameter. The non-epithelialised diameter is subtracted from the total wound diameter, to determine the distance which the new epithelium has covered since the time of wounding. Percentage re-epithelialisation for the new epithelium was calculated as a proportion of the total wound diameter. The average percentage re-epithelialisation value was calculated from the 44 wounds of each treatment group.

### 2.2 Results

The results of this study are shown in FIG. 2.

This illustrates that all concentrations of the medicaments of the invention tested were able to accelerate re-epithelialisation (and hence wound healing) as compared to placebo controls.

The medicament comprising 0.2 μg 17β-oestradiol/1001 (0.0002%) of medicament was most effective, and wounds treated with this medicament exhibited 42.6% re-epithelialisation three days after wounding. In contrast wounds treated with the medicament comprising 0.1 μg 17ρ-oestradiol/100 μl (0.0001%) exhibited 42.3% re-epithelialisation three days after wounding, and those treated with the medicament comprising 0.02 μg 17β-oestradiol/100 μl (0.00002%) exhibited 37.4% re-epithelialisation three days after wounding.

By way of contrast, wounds treated with the placebo control exhibited 36.6% re-epithelialisation three days after wounding,

### 3 Acceleration of Healing of Partial Thickness Wounds Using Medicaments of the Invention.

The study described here illustrates that medicaments of the invention are able to therapeutically effectively accelerate healing of partial thickness wounds (which provide a model of split thickness skin graft donor sites), and are able to do so when incorporating lower concentrations of a compound that promotes oestrogenic activity than would be suggested by the teachings of the prior art.

### 3.1 Materials and Methods

### 3.1.1 Medicaments of the Invention

Medicaments of the invention incorporating a range of test concentrations of 17β-oestradiol (a compound that promotes oestrogenic activity) were prepared using the constituents described above.

Two test concentrations of the medicaments were prepared as follows:
i) 0.1 μg 17β-oestradiol/100 μl of medicament (0.0001%)
ii) 0.2 μg 17β-oestradiol/100 μl of medicament (0.0002%)

### 3.1.2 Wound Healing Model

A pig partial thickness wound healing model was used to assess the ability of the medicaments of the invention to accelerate the healing of wounds providing a model split thickness skin graft donor sites.

Test medicaments or diluent control (PBS and 5% v/v ethanol without 17β-oestradiol) were administered by intradermal injection to provide 100 μl of medicament per cm² to sites on experimental pigs where wounds were to be formed. Thus a total volume of 625 μl of the test medicament or diluent control was administered per wound site. After administration of the medicaments or diluent control, 2.5 cm by 2.5 cm (6.25 cm²) partial thickness wounds were made using a 25 mm dermatome (Nouvag) at a depth setting of 0.55 mm. A single wound was treated per animal, and each treatment group comprised four male Large White pigs.

### 3.1.3 Assessment of Acceleration of Wound Healing

Animals were killed, and their wounds harvested, five days after wounding. The wounds were excised and treated for histology. Histological sections from wounds treated with medicaments of the invention and from wounds treated with diluent control were studied and the percentage of re-epithelialisation exhibited by the different wounds calculated and compared. After being fixed for 48 hours wounded tissue was bisected into four even sections. All bisected sections were processed to wax and embedded into individual wax blocks. Tissue was sectioned through to the end of the block where at every 250 μm, a single slide containing a 5 μm thick section was prepared. Each section was stained with Haematoxylin and Eosin to aid visualisation of structural features, and measurements made using image analysis software. For each section, the freehand epithelialised and freehand non-epithelialised distances were measured using image analysis software; freehand epithelialised distance as a proportion of the sum of the epithelialised and non-epithelialised distances was calculated to produce a percentage re-epithelialisation value. Average percentage re-epithelialisation values were calculated per wound and per treatment group.

### 3.2 Results

The results of this study are shown in FIG. 3.

As can be seen from FIG. 3, a statistically significant increase in re-epithelialisation of partial thickness wounds was observed in respect of all concentrations of the medicaments investigated. Wounds treated with the medicaments of the invention exhibited greater than 98% re-epithelialisation.

The medicaments of the invention found to most effectively promote re-epithelialisation, and hence wound healing, contain concentrations of 17β-oestradiol (constituting the required compound that promotes oestrogenic activity) at concentrations that are five or ten times lower than the minimum effective concentrations suggested by the prior art.

1. The use of a compound that promotes oestrogenic activity in the manufacture of a medicament for administration in an amount to provide per cm² of the administered area a level of oestrogenic activity equivalent to that provided by up to 300 ng of 17β-oestradiol, for accelerating the healing of wounds.

2. The use according to claim 1, wherein the medicament is for administration in an amount to provide per cm2 of the administered area a level of oestrogenic activity equivalent to that provided by between 1 ng and 300 ng of 17β-oestradiol.

3. The use according to claim 2, wherein the medicament is for administration in an amount to provide per cm² of the administered area a level of oestrogenic activity equivalent to that provided by between 20 ng and 300 ng of 17β-oestradiol.

4. The use according to claim 3, wherein the medicament is for administration in an amount to provide per cm² of the administered area a level of oestrogenic activity equivalent to that provided by between 100 ng and 200 ng of 17β-oestradiol.

5. The use according to claim 4, wherein the medicament is for administration in an amount to provide per cm² of the administered area a level of oestrogenic activity equivalent to that provided by approximately 100 ng of 17β-oestradiol.

6. The use according to claim 5, wherein the medicament is for administration in an amount to provide per cm² of the administered area a level of oestrogenic activity equivalent to that provided by approximately 200 ng of 17β-oestradiol.

7. The use according to claim 1, wherein the compound that promotes oestrogenic activity is selected from the group consisting of oestrogens; oestrogen receptor agonists such as ethinylyoestradiol, dienoestrol, mestranol, oestradiol, oestriol, conjugated oestrogens, piperazine oestrone sulphate, stilboestrol, fosfesterol tetrasodium, polyestradiol phosphate and tibolone; inhibitors of oestrogen or oestrogen receptor agonist breakdown; phytoestrogens; modulators of luteinising hormone; chorionic gonadotrophin, and 17β-oestradiol.

8. The use according to claim 7, wherein the compound that promotes oestrogenic activity is 17β-oestradiol.

9. The use according to claim 1, wherein the medicament is a topical medicament.

10. The use according to claim 9, wherein the topical medicament is selected from the group consisting of a topical injectable solution; an irrigation solution; a topical spray; a topical cream; and a wound dressing, including a foam or sponge dressing, or a film dressing.

11. The use according to claim 1, wherein the medicament is for the healing of skin wounds.

12. The use according to claim 11, wherein the skin wound is associated with a skin peel.

13. The use according to claim 1, wherein the medicament is for the healing of corneal wounds.

14. The use according to claim 1, wherein the medicament is administered prior to wound formation.

15. The use according to claim 1, wherein the medicament is administered after wound formation.

16. The use according to claim 1, wherein the medicament is formulated to provide discrete dosage units capable of providing the specified amount of oestrogenic activity.

17. A method of accelerating the healing of a wound, the method comprising administering to a site in need of such accelerated healing a therapeutically effective amount of a compound that promotes oestrogenic activity, the therapeutically effective amount being an amount that provides oestrogenic activity per cm² administered equivalent to that produced by up to 300 ng of 17β-oestradiol.

18. The method according to claim 17, wherein the therapeutically effective amount of the compound that promotes oestrogenic activity is an amount that provides oestrogenic activity per centimetre of wound being treated equivalent to that produced by between 1 ng and 300 ng of 17β-oestradiol.

19. The method according to claim 18, wherein the therapeutically effective amount of the compound that promotes oestrogenic activity is an amount that provides oestrogenic activity per centimetre of wound being treated equivalent to that produced by between 20 ng and 300 ng of 17β-oestradiol.

20. The method according to claim 19, wherein the therapeutically effective amount of the compound that promotes oestrogenic activity is an amount that provides oestrogenic activity per centimetre of wound being treated equivalent to that produced by between 100 ng and 200 ng of 17β-oestradiol.

21. The method according to claim 20, wherein the therapeutically effective amount of the compound that promotes oestrogenic activity is an amount that provides oestrogenic activity per centimetre of wound being treated equivalent to that produced by 100 ng of 17β-oestradiol.

22. The method according to claim 21, wherein the therapeutically effective amount of the compound that promotes oestrogenic activity is an amount that provides oestrogenic activity per centimetre of wound being treated equivalent to that produced by 200 ng of 17β-oestradiol.

23. The method according to claim 17 wherein the site in need of accelerated healing is a site where a wound is to be formed.

24. The method according to claim 17, wherein the site in need of accelerated healing is a wound.

25. The method according to claim 24, wherein the wound is a full thickness wound.

26. The method according to claim 24, wherein the wound is a partial thickness wound.

27. The method according to claim 16, wherein the site in need of accelerated healing is the skin.

28. The method according to claim 16, wherein the compound that promotes oestrogenic activity is 17β-oestradiol.
